(19) United States
(12) Patent Application Publication
(10) Pub. No.: US 2022/0372033 A1
(43) Pub. Date: Nov. 24, 2022

(54) ANTIBACTERIAL COMPOUNDS

(71) Applicant: Janssen Sciences Ireland Unlimited Company, Co. Cork (IE)

(72) Inventors: Jérôme Émile Georges GUILLEMONT, Andé (FR); Steffen Friedrich Walter WEIDNER, Illkirch-Graffenstaden (FR); Ellen Anita LANCKACKER, Lochristi (BE); Godelieve Maria J. LAMMENS, Geel (BE); Dirk Antonie LAMPRECHT, Turnhout (BE)

(21) Appl. No.: 17/764,760

(22) PCT Filed: Sep. 29, 2020
(86) PCT No.: PCT/EP2020/077175

§ 371 Date: Mar. 29, 2022

(30) Foreign Application Priority Data
Sep. 30, 2019 (EP) 19200377.0

(51) Int. Cl. C07D 471/04; A61K 38/00; A61P 31/04

(52) U.S. Cl. CPC C07D 471/04; A61K 38/005; A61P 31/04

(57) ABSTRACT

The present invention relates to the following compounds (I) wherein the integers are as defined in the description, and where the compounds may be useful as medicaments, for instance for use in the treatment of tuberculosis (e.g. in combination).

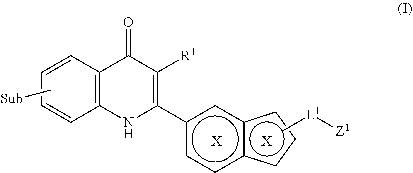

The present invention relates to novel compounds. The invention also relates to such compounds for use as a pharmaceutical and further for the use in the treatment of bacterial diseases, including diseases caused by pathogenic mycobacteria such as Mycobacterium tuberculosis. Such compounds may work by targeting the respiratory chain, and thereby blocking all energy production of mycobacteria. There are several ways of targeting the electron transport chain of mycobacteria, for instance by interfering with ATP synthase in M. tuberculosis. This particular invention focuses on the cytochrome bd target of the respiratory chain, which may be the primary mode of action. Hence, primarily, such compounds are antitubercular agents, and in particular may act as such when combined with another tuberculosis drug (e.g. another inhibitor of a different target of the electron transport chain).

### BACKGROUND OF THE INVENTION

Mycobacterium tuberculosis is the causative agent of tuberculosis (TB), a serious and potentially fatal infection with a world-wide distribution. Estimates from the World Health Organization indicate that more than 8 million people contract TB each year, and 2 million people die from tuberculosis yearly. In the last decade, TB cases have grown 20% worldwide with the highest burden in the most impoverished communities. If these trends continue, TB incidence will increase by 41% in the next twenty years. Fifty years since the introduction of an effective chemotherapy, TB remains after AIDS, the leading infectious cause of adult mortality in the world. Complicating the TB epidemic is the rising tide of multi-drug-resistant strains, and the deadly symbiosis with HIV. People who are HIV-positive and infected with TB are 30 times more likely to develop active TB than people who are HIV-negative and TB is responsible for the death of one out of every three people with HIV/AIDS worldwide.

Existing approaches to treatment of tuberculosis all involve the combination of multiple agents. For example, the regimen recommended by the U.S. Public Health Service is a combination of isoniazid, rifampicin and pyrazinamide for two months, followed by isoniazid and rifampicin alone for a further four months. These drugs are continued for a further seven months in patients infected with HIV. For patients infected with multi-drug resistant strains of M. tuberculosis, agents such as ethambutol, streptomycin, kanamycin, amikacin, capreomycin, ethionamide, cycloserine, ciprofoxacin and ofloxacin are added to the combination therapies. There exists no single agent that is effective in the clinical treatment of tuberculosis, nor any combination of agents that offers the possibility of therapy of less than six months' duration.

There is a high medical need for new drugs that improve current treatment by enabling regimens that facilitate patient and provider compliance. Shorter regimens and those that require less supervision are the best way to achieve this. Most of the benefit from treatment comes in the first 2 months, during the intensive, or bactericidal, phase when four drugs are given together; the bacterial burden is greatly reduced, and patients become noninfectious. The 4- to 6-month continuation, or sterilizing, phase is required to eliminate persisting bacilli and to minimize the risk of relapse. A potent sterilizing drug that shortens treatment to 2 months or less would be extremely beneficial. Drugs that facilitate compliance by requiring less intensive supervision also are needed. Obviously, a compound that reduces both the total length of treatment and the frequency of drug administration would provide the greatest benefit.

Complicating the TB epidemic is the increasing incidence of multi-drug-resistant strains or MDR-TB. Up to four percent of all cases worldwide are considered MDR-TB —those resistant to the most effective drugs of the four-drug standard, isoniazid and rifampin. MDR-TB is lethal when untreated and cannot be adequately treated through the standard therapy, so treatment requires up to 2 years of “second-line” drugs. These drugs are often toxic, expensive and marginally effective. In the absence of an effective therapy, infectious MDR-TB patients continue to spread the disease, producing new infections with MDR-TB strains. There is a high medical need for a new drug with a new mechanism of action, which is likely to demonstrate activity against drug resistant, in particular MDR strains.

The term “drug resistant” as used hereinbefore or hereinafter is a term well understood by the person skilled in microbiology. A drug resistant Mycobacterium is a Mycobacterium which is no longer susceptible to at least one previously effective drug; which has developed the ability to withstand antibiotic attack by at least one previously effective drug. A drug resistant strain may relay that ability to withstand to its progeny. Said resistance may be due to random genetic mutations in the bacterial cell that alters its sensitivity to a single drug or to different drugs.

MDR tuberculosis is a specific form of drug resistant tuberculosis due to a bacterium resistant to at least isoniazid and rifampicin (with or without resistance to other drugs), which are at present the two most powerful anti-TB drugs. Thus, whenever used hereinbefore or hereinafter “drug resistant” includes multi drug resistant.

Another factor in the control of the TB epidemic is the problem of latent TB. In spite of decades of tuberculosis (TB) control programs, about 2 billion people are infected by M. tuberculosis, though asymptomatically. About 10% of these individuals are at risk of developing active TB during their lifespan. The global epidemic of TB is fuelled by infection of HIV patients with TB and rise of multi-drug resistant TB strains (MDR-TB). The reactivation of latent TB is a high risk factor for disease development and accounts for 32% deaths in HIV infected individuals. To control TB epidemic, the need is to discover new drugs that can kill dormant or latent bacilli. The dormant TB can get reactivated to cause disease by several factors like suppression of host immunity by use of immunosuppressive agents like antibodies against tumor necrosis factor α or interferon-γ. In case of HIV positive patients the only prophylactic treatment available for latent TB is two-three months regimens of rifampicin, pyrazinamide. The efficacy of the treatment regime is still not clear and furthermore the length of the treatments is an important constrain in resource-limited environments. Hence there is a drastic need to identify new drugs, which can act as chemoprophylatic agents for individuals harboring latent TB bacilli.

The tubercle bacilli enter healthy individuals by inhalation; they are phagocytosed by the alveolar macrophages of the lungs. This leads to potent immune response and formation of granulomas, which consist of macrophages infected with M. tuberculosis surrounded by T cells. After a period of 6-8 weeks the host immune response cause death of infected cells by necrosis and accumulation of caseous material with certain extracellular bacilli, surrounded by macrophages, epitheloid cells and layers of lymphoid tissue at the periphery. In case of healthy individuals, most of the mycobacteria are killed in these environments but a small proportion of bacilli still survive and are thought to exist in a non-replicating, hypometabolic state and are tolerant to killing by anti-TB drugs like isoniazid. These bacilli can remain in the altered physiological environments even for individual's lifetime without showing any clinical symptoms of disease. However, in 10% of the cases these latent bacilli may reactivate to cause disease. One of the hypothesis about development of these persistent bacteria is patho-physiological environment in human lesions namely, reduced oxygen tension, nutrient limitation, and acidic pH. These factors have been postulated to render these bacteria phenotypically tolerant to major anti-mycobacterial drugs.

In addition to the management of the TB epidemic, there is the emerging problem of resistance to first-line antibiotic agents. Some important examples include penicillin-resistant Streptococcus pneumoniae, vancomycin-resistant enterococci, methicillin-resistant Staphylococcus aureus, multi-resistant salmonellae.

The consequences of resistance to antibiotic agents are severe. Infections caused by resistant microbes fail to respond to treatment, resulting in prolonged illness and greater risk of death. Treatment failures also lead to longer periods of infectivity, which increase the numbers of infected people moving in the community and thus exposing the general population to the risk of contracting a resistant strain infection. Hospitals are a critical component of the antimicrobial resistance problem worldwide. The combination of highly susceptible patients, intensive and prolonged antimicrobial use, and cross-infection has resulted in infections with highly resistant bacterial pathogens.

Self-medication with antimicrobials is another major factor contributing to resistance. Self-medicated antimicrobials may be unnecessary, are often inadequately dosed, or may not contain adequate amounts of active drug.

Patient compliance with recommended treatment is another major problem. Patients forget to take medication, interrupt their treatment when they begin to feel better, or may be unable to afford a full course, thereby creating an ideal environment for microbes to adapt rather than be killed.

Because of the emerging resistance to multiple antibiotics, physicians are confronted with infections for which there is no effective therapy. The morbidity, mortality, and financial costs of such infections impose an increasing burden for health care systems worldwide.

Therefore, there is a high need for new compounds to treat bacterial infections, especially mycobacterial infections including drug resistant and latent mycobacterial infections, and also other bacterial infections especially those caused by resistant bacterial strains.

There are several ways of targeting the electron transport chain of mycobacteria, for instance by interfering with ATP synthase in M. tuberculosis. Unlike many bacteria, M. tuberculosis is dependent on respiration to synthesise adequate amounts of ATP. Hence targeting the electron transport chain of the mycobacteria and thereby blocking energy production of mycobacteria is thought to be a potentially effective way of providing an efficient regimen against mycobacteria. Targets already known are ATP synthase inhibitors, as example of which is bedaquiline (marketed as Sirturo®), cytochrome be inhibitors, examples of which include the compound Q203 described in Journal article Nature Medicine, 19, 1157-1160 (2013) by Pethe et al “Discovery of Q203, a potent clinical candidate for the treatment of tuberculosis”, as well as patent applications such as international patent applications WO 2017/001660, WO 2017/001661, WO 2017/216281 and WO 2017/216283.

Additionally, journal article Antimicrob. Agents Chemother, 2014, 6962-6965 by Arora et al describes compounds that target the respiratory bc₁ complex in M. tuberculosis, and where deletion of the cytochrome bd oxidase generated a hypersusceptible mutant. Journal article PANS (Early Edition), 2017, “Exploiting the synthetic lethality between terminal respiratory oxidases to kill Mycobacterium tuberculosis and clear host infection” by Kalia et al discloses various data around various tuberculosis compounds that target the respiratory chain. For instance, it is shown that the compound Q203 (a known bc inhibitor; see above) could inhibit mycobacteria completely and become bactericidal, after genetic deletion of the cytochrome bd oxidase-encoding genes CydAB. Similarly, journal article MBio, 2014 Jul. 15; 5(4) by Berney et al “A Mycobacterium tuberculosis cytochrome bd oxidase mutant is hypersensitive to bedaquiline” shows that the activity of bedaquiline is enhanced when bd is inactivated.

One known cytochrome bd inhibitor is Aurachin D, which is a quinolone with a relatively long side-chain. Cytochrome bd itself is not essential for aerobic growth, but is upregulated and protects against a variety of stresses in various bacterial strains, for example as described in journal article Biochimica et Biophysica Acta 1837 (2014) 1178-1187 by Giuffre et al. Hence, monotherapy with a cytochrome bd inhibitor would not necessarily be expected to inhibit mycobacteria growth, but a combination with another inhibitor of a target of the electron transport chain of mycobacteria could be.

Various compounds are described in international patent applications WO 2012/069856 and WO 2017/103615, with the latter application describing such compounds as cytochrome bd inhibitors and indicates that therapeutic combination products comprising one or more respiratory electron transport chain inhibitor and a cytochrome bd inhibitor is disclosed. Specifically, the compound CK-2-63 is described as a cytochrome bd inhibitor showing various inhibitor activity data, and combination data is also disclosed including combination of CK-2-63 with a mycobacterium cytochrome bcc inhibitor (e.g. AWE-402, where it is indicated therein that it is structurally related to the cytochrome bcc inhibitor Q203). It is indicated that such dual combination led to in increase in mycobacteria kill. It also described a combination of bedaquiline (a known ATP synthase inhibitor) with CK-2-63, and it is indicated that CK-2-63 showed an enhancement of bedaquiline activity at low concentrations. Data around a triple combination of bedaquiline, AWE-402 (a be inhibitor; see above) and CK-2-63 is also shown.

This particular invention focuses on novel compounds of the cytochrome bd target of the respiratory chain. New alternative/improved compounds are required, which may be tested/employed for use in combination.

### SUMMARY OF THE INVENTION

There is now provided a compound of formula (I)

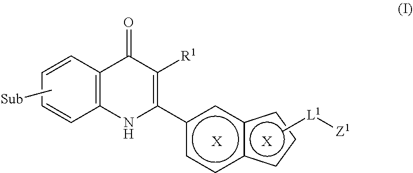

wherein

R¹ represents C₁₋₆ alkyl, —Br, hydrogen or —C(O)N(R^{q1})R^{q2};

R^{q1} and R^{q2} independently represent hydrogen or C₁₋₆ alkyl, or may be linked together to form a 3-6 membered carbocylic ring optionally substituted by one or more C₁₋₃ alkyl substituents;

Sub represents one or more optional substituents selected from halo, —CN, C₁₋₆ alkyl and —O—C₁₋₆ alkyl (wherein the latter two alkyl moieties are optionally substituted by one or more fluoro atoms);

the two “X” rings together represent a 9-membered bicyclic heteroaryl ring (consisting of a 6-membered aromatic ring fused to another 5-membered aromatic ring), which bicyclic heteroaryl ring contains between one and four heteroatoms (e.g. selected from nitrogen, oxygen and sulfur), and which bicyclic ring is optionally substituted by one or more substituents selected from halo and C₁₋₆ alkyl (itself optionally substituted by one or more fluoro atoms);

L¹ represents an optional linker group, and hence may be a direct bond, —O—, —OCH₂—, —C(R^{x1})(R^{x2})— or —C(O)—N(H)—CH₂—;

R^{x1} and R^{x2} independently represent hydrogen or C₁₋₃ alkyl;

Z¹ represents either one of the following moieties:

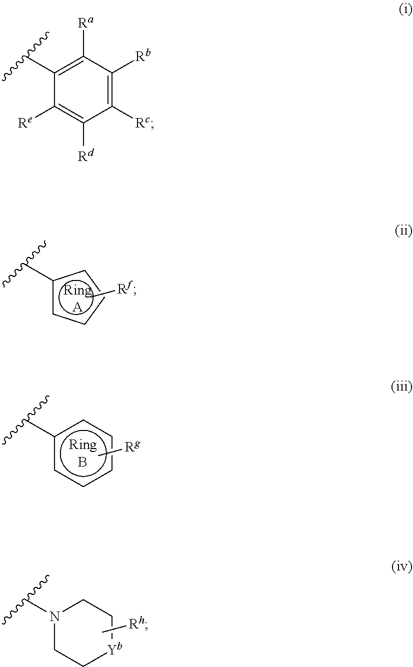

(v) perfluoro C₁₋₃ alkyl (e.g. —CF₃);
(vi) —F, —Br, —Cl or —CN;

ring A represents a 5-membered aromatic ring containing at least one heteroatom (preferably containing at least one nitrogen atom), and which ring is optionally substituted by one or more substituents independently selected from R^{f};

ring B represents a 6-membered aromatic ring containing at least one heteroatom (preferably containing at least one nitrogen atom), and which ring is optionally substituted by one or more substituents independently selected from R^{g};

Y^{b} represents —CH₂ or NH, and R^{h} represents one or more substituents on the 6-membered N and Y^{b}-containing ring (which R^{h} substituents may also be present on Y^{b});

R^{a}, R^{b}, R^{c}, R^{d} and R^{e} independently represent hydrogen or a substituent selected from B¹;

each R^{f}, each R^{g} and each R^{h} (which are optional substituents), when present, independently represent a substituent selected from B¹;

each B¹ independently represents a substituent selected from:
(i) halo;
(ii) —R^{d1};
(iii) —OR^{e1};
(iv) —C(O)N(R^{e2})R^{e3}
(v) —SF₅;
(vi) —N(R^{e4})S(O)₂R^{e5};

R^{d1} represents C₁₋₆ alkyl optionally substituted by one or more halo (e.g. fluoro) atoms;

R^{e1}, R^{e2}, R^{e3}, R^{e4} and R^{e5} each independently represent hydrogen or C₁₋₆ alkyl optionally substituted by one or more fluoro atoms;

or a pharmaceutically-acceptable salt thereof,

which compounds may be referred to herein as “compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable acid addition salts as mentioned hereinabove are meant to comprise the therapeutically active non-toxic acid addition salt forms that the compounds of formula (I) are able to form. These pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

For the purposes of this invention solvates, prodrugs, N-oxides and stereoisomers of compounds of the invention are also included within the scope of the invention.

The term “prodrug” of a relevant compound of the invention includes any compound that, following oral or parenteral administration, is metabolised in vivo to form that compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of compounds of the invention may be prepared by modifying functional groups present on the compound in such a way that the modifications are cleaved, in vivo when such prodrug is administered to a mammalian subject. The modifications typically are achieved by synthesising the parent compound with a prodrug substituent. Prodrugs include compounds of the invention wherein a hydroxyl, amino, sulfhydryl, carboxy or carbonyl group in a compound of the invention is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, New York-Oxford (1985).

Compounds of the invention may contain double bonds and may thus exist as E (entgegen) and Z (zusammen) geometric isomers about each individual double bond. Positional isomers may also be embraced by the compounds of the invention. All such isomers (e.g. if a compound of the invention incorporates a double bond or a fused ring, the cis- and trans-forms, are embraced) and mixtures thereof are included within the scope of the invention (e.g. single positional isomers and mixtures of positional isomers may be included within the scope of the invention).

Compounds of the invention may also exhibit tautomerism. All tautomeric forms (or tautomers) and mixtures thereof are included within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerisations. Valence tautomers include interconversions by reorganisation of some of the bonding electrons.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person.

All stereoisomers (including but not limited to diastereoisomers, enantiomers and atropisomers) and mixtures thereof (e.g. racemic mixtures) are included within the scope of the invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

The compounds of the present invention may exist in unsolvated as well as solvated forms with pharmaceutically acceptable solvents such as water, ethanol, and the like, and it is intended that the invention embrace both solvated and unsolvated forms.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, 17O, 18O, ³²P, ³³P, ³⁵S, ¹⁸F, ³⁶Cl, ¹²³I, and ¹²⁵I. Certain isotopically-labeled compounds of the present invention (e.g., those labeled with ³H and ¹⁴C) are useful in compound and for substrate tissue distribution assays. Tritiated (³H) and carbon-14 (¹⁴C) isotopes are useful for their ease of preparation and detectability. Further, substitution with heavier isotopes such as deuterium (i.e., ²H may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the description/Examples hereinbelow, by substituting an isotopically labeled reagent for a non-isotopically labeled reagent.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group). Such cycloalkyl groups may be monocyclic or bicyclic and may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q} alkynyl group).

C_{3-q} cycloalkyl groups (where q is the upper limit of the range) that may be specifically mentioned may be monocyclic or bicyclic alkyl groups, which cycloalkyl groups may further be bridged (so forming, for example, fused ring systems such as three fused cycloalkyl groups). Such cycloalkyl groups may be saturated or unsaturated containing one or more double bonds (forming for example a cycloalkenyl group). Substituents may be attached at any point on the cycloalkyl group. Further, where there is a sufficient number (i.e. a minimum of four) such cycloalkyl groups may also be part cyclic.

The term “halo”, when used herein, preferably includes fluoro, chloro, bromo and iodo.

Heterocyclic groups when referred to herein may include aromatic or non-aromatic heterocyclic groups, and hence encompass heterocycloalkyl and hetereoaryl. Equally, “aromatic or non-aromatic 5- or 6-membered rings” may be heterocyclic groups (as well as carbocyclic groups) that have 5- or 6-members in the ring.

Heterocycloalkyl groups that may be mentioned include non-aromatic monocyclic and bicyclic heterocycloalkyl groups in which at least one (e.g. one to four) of the atoms in the ring system is other than carbon (i.e. a heteroatom), and in which the total number of atoms in the ring system is between 3 and 20 (e.g. between three and ten, e.g between 3 and 8, such as 5- to 8-). Such heterocycloalkyl groups may also be bridged. Further, such heterocycloalkyl groups may be saturated or unsaturated containing one or more double and/or triple bonds, forming for example a C_{2-q} heterocycloalkenyl (where q is the upper limit of the range) group. C_{2-q} heterocycloalkyl groups that may be mentioned include 7-azabicyclo[2.2.1]heptanyl, 6-azabicyclo[3.1.1]heptanyl, 6-azabicyclo[3.2.1]-octanyl, 8-azabicyclo-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo-[3.2.1]octanyl, oxetanyl, oxiranyl, piperazinyl, piperidinyl, non-aromatic pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form. Heterocycloalkyl mentioned herein may be stated to be specifically monocyclic or bicyclic.

Aromatic groups may be aryl or heteroaryl. Aryl groups that may be mentioned include C₆₋₂₀, such as C₆₋₁₂ (e.g. C₆₋₁₀) aryl groups. Such groups may be monocyclic, bicyclic or tricyclic and have between 6 and 12 (e.g. 6 and 10) ring carbon atoms, in which at least one ring is aromatic. C₆₋₁₀ aryl groups include phenyl, naphthyl and the like, such as 1,2,3,4-tetrahydronaphthyl. The point of attachment of aryl groups may be via any atom of the ring system. For example, when the aryl group is polycyclic the point of attachment may be via atom including an atom of a non-aromatic ring. However, when aryl groups are polycyclic (e.g. bicyclic or tricyclic), they are preferably linked to the rest of the molecule via an aromatic ring. Most preferred aryl groups that may be mentioned herein are “phenyl”.

Unless otherwise specified, the term “heteroaryl” when used herein refers to an aromatic group containing one or more heteroatom(s) (e.g. one to four heteroatoms) preferably selected from N, O and S. Heteroaryl groups include those which have between 5 and 20 members (e.g. between 5 and 10) and may be monocyclic, bicyclic or tricyclic, provided that at least one of the rings is aromatic (so forming, for example, a mono-, bi-, or tricyclic heteroaromatic group). When the heteroaryl group is polycyclic the point of attachment may be via any atom including an atom of a non-aromatic ring. However, when heteroaryl groups are polycyclic (e.g. bicyclic or tricyclic), they are preferably linked to the rest of the molecule via an aromatic ring. Heteroaryl groups that may be mentioned include 3,4-dihydro-1H-isoquinolinyl, 1,3-dihydroisoindolyl, 1,3-dihydroisoindolyl (e.g. 3,4-dihydro-1H-isoquinolin-2-yl, 1,3-dihydroisoindol-2-yl, 1,3-dihydroisoindol-2-yl; i.e. heteroaryl groups that are linked via a non-aromatic ring), or, preferably, acridinyl, benzimidazolyl, benzodioxanyl, benzodioxepinyl, benzo-dioxolyl (including 1,3-benzodioxolyl), benzofuranyl, benzofurazanyl, benzothiadiazolyl (including 2,1,3-benzothiadiazolyl), benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetra-hydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Heteroaryl groups mentioned herein may be stated to be specifically monocyclic or bicyclic. When heteroaryl groups are polycyclic in which there is a non-aromatic ring present, then that non-aromatic ring may be substituted by one or more ═O group. Most preferred heteroaryl groups that may be mentioned herein are 5- or 6-membered aromatic groups containing 1, 2 or 3 heteroatoms (e.g. preferably selected from nitrogen, oxygen and sulfur).

It may be specifically stated that the heteroaryl group is monocyclic or bicyclic. In the case where it is specified that the heteroaryl is bicyclic, then it may consist of a five-, six- or seven-membered monocyclic ring (e.g. a monocyclic heteroaryl ring) fused with another five-, six- or seven-membered ring (e.g. a monocyclic aryl or heteroaryl ring).

Heteroatoms that may be mentioned include phosphorus, silicon, boron and, preferably, oxygen, nitrogen and sulfur.

When “aromatic” groups are referred to herein, they may be aryl or heteroaryl. When “aromatic linker groups” are referred to herein, they may be aryl or heteroaryl, as defined herein, are preferably monocyclic (but may be polycyclic) and attached to the remainder of the molecule via any possible atoms of that linker group. However, when, specifically carbocylic aromatic linker groups are referred to, then such aromatic groups may not contain a heteroatom, i.e. they may be aryl (but not heteroaryl).

For the avoidance of doubt, where it is stated herein that a group may be substituted by one or more substituents (e.g. selected from C₁₋₆ alkyl), then those substituents (e.g. alkyl groups) are independent of one another. That is, such groups may be substituted with the same substituent (e.g. same alkyl substituent) or different (e.g. alkyl) substituents.

All individual features (e.g. preferred features) mentioned herein may be taken in isolation or in combination with any other feature (including preferred feature) mentioned herein (hence, preferred features may be taken in conjunction with other preferred features, or independently of them).

The skilled person will appreciate that compounds of the invention that are the subject of this invention include those that are stable. That is, compounds of the invention include those that are sufficiently robust to survive isolation from e.g. a reaction mixture to a useful degree of purity.

Preferred compounds of the invention include those in which:

when R¹ represents —C(O)N(R^{q1})R^{q2}, then R^{q1} and R^{q2} independently represent hydrogen C₁₋₃ alkyl (so forming e.g. —C(O)N(H)CH₃ or —C(O)N(CH₃)₂);

R¹, in an embodiment, represents hydrogen, C₁₋₆ alkyl or —C(O)N(R^{q1})R^{q2};

one of R^{q1} and R^{q2} represents C₁₋₃ alkyl (e.g. methyl) and the other represents hydrogen or C₁₋₃ alkyl (e.g. methyl);

R¹, in a further embodiment, represents C₁₋₆ alkyl, e.g. C₁₋₃ alkyl such as methyl;

Sub is not present, i.e. there are no further substituents on the relevant aromatic/benzene ring, or represents one or two substituents selected from halo (e.g. fluoro and/or chloro) and —OC₁₋₃ alkyl (e.g. —OCH₃).

Compounds of the invention contain a 9-membered bicyclic heteroaromatic group represented by the “X” rings. In an embodiment, further compounds of the invention include those in which such bicyclic ring:

contains at least one nitrogen atom (in an embodiment, at the ring junction); and/or contains one, two, three or four heteroatoms in total; and/or

in addition to being substituted by L¹, is optionally further substituted by one or two (e.g. one) further substituent selected from C₁₋₃ alkyl and —OC₁₋₃ alkyl (in which the latter two alkyl moieties are each optionally substituted with fluoro, so forming e.g. a —CF₃, —OCF₃ or —OCH₃ substituent).

In an embodiment of the invention, compounds of the invention are those in which the “X” rings (the bicyclic heteroaryl group) is represented by a sub-formula (IA) as defined hereinbelow (where it will be appreciated that the rules of valency will be adhered to, e.g. where C is mentioned then it may need to have a H attached to it), in which:

one of X¹ and X² represents N (i.e. there is an essential nitrogen at the ring junction) and the other represents C;

the other integers X³, X⁴ and X⁵ may represent C (or CH) or a heteroatom (such as N, O and/or S); and/or

none, any one or two of X³, X⁴ and X⁵ represents a heteroatom (e.g. N, O and/or S) and the other represents C (or CH).

The “X” rings in compounds of the invention (the 9-membered bicyclic heteroaryl group) may be depicted as follows (in which the left hand side would be further bound to the requisite quinolinone or formula (I) and the right hand side would be further bound to the Lⁱ group of formula (I):

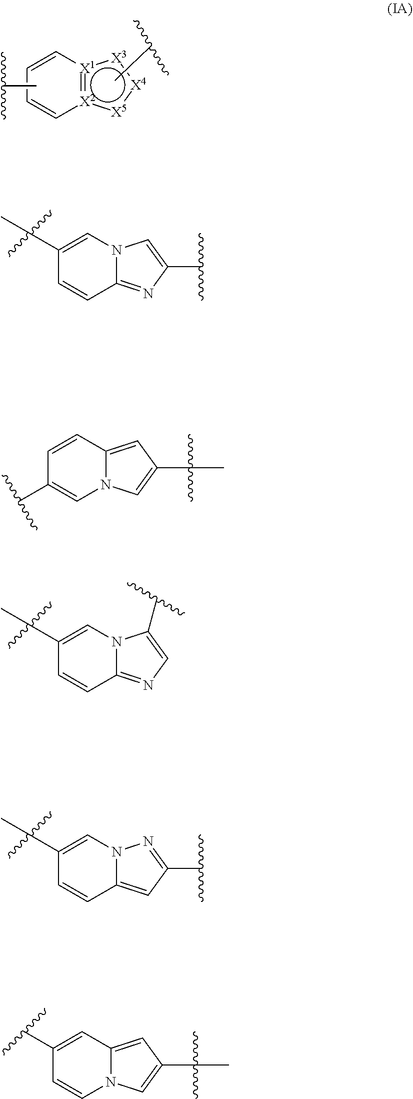

In a further embodiment, preferred compounds of the invention include those in which in the sub-formula (IA) depicted above:

any three of X¹, X², X³, X⁴ and X⁵ represent a heteroatom (e.g. nitrogen) and the other two represent C (or CH);

one of X¹ and X² represents N (i.e. there is an essential nitrogen at the ring junction) and the other represents C;

none, any one or any two of X³, X⁴ and X⁵ represents a N heteroatom and the other represents C (or CH); and/or

the 9-membered bicyclic heteroaryl group depicted by X is as defined in the formulae above,

and in which in all of the cases above, it will be understood that the rules of valency will need to be adhered to.

Other preferred compounds of the invention include those in which:

L¹ represents a direct bond, —O—, —OCH₂—-C(R^{x1})(R^{x2})— or —C(O)—N(H)—CH₂—;

R^{x1} and R^{x2} independently represent hydrogen; for example:

L¹ may specifically represent a direct bond, —O—, —OCH₂— or —CH₂— (or, in a more specific embodiment, a direct bond, —O— or —CH₂—; especially a direct bond or —CH₂—).

In embodiments of the invention Z¹ represents:

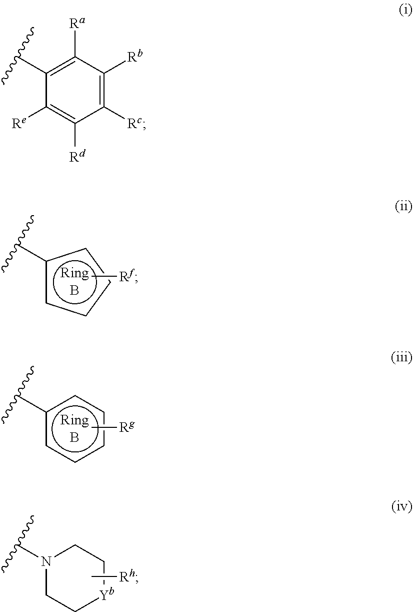

(v) perfluoro C₁₋₃ alkyl (e.g. —CF₃); or
(vi) —F, —Br, —Cl or —CN;

and hence there are six embodiments of the invention, and in an aspect, Z¹ represents (i), (ii) or (iii) (e.g. Z¹ represents (i) or (ii)) and, in a further aspect, Z¹ represents (iv) and, in a separate embodiment, Z¹ represents (v) or (vi) (e.g. Z¹ represents (v)). Hence, in an embodiment, Z¹ represents an aromatic ring (i.e. (i), (ii) or (iii) above), for instance (i) or (ii).

In a further embodiment, compounds of the invention include those in which

when ring A is present, it represents a 5-membered aromatic ring, it contains one, two or three heteroatoms preferably selected from nitrogen, oxygen and sulfur; in a further embodiment, such ring is optionally substituted by one or two substituents independently selected from R;

when ring B is present, it represents a 6-membered aromatic ring containing one nitrogen atom; and, in a further embodiment, such ring is optionally substituted by one or two substituents independently selected from R^{g};

Y^{b} represents —CH₂ or NH, and R^{h} represents one or two substituents on the 6-membered N and Y^{b}-containing ring (which R^{h} substituents may also be present on Y^{b});

R^{a}, R^{b}, R^{c}, R^{d} and R^{e} independently represent hydrogen or a substituent selected from B¹;

R^{f}, R^{g} and R^{h} each independently represent a substituent selected from B¹.

In an embodiment, when Ring A is present (i.e. Z¹ represents (ii)), then such aromatic 5-membered (optionally substituted) ring may: (i) contain one sulfur atom (so forming a thienyl); (ii) contain one nitrogen and one sulfur atom (so forming e.g. thiazolyl); (iii) contain two nitrogen atoms (so forming e.g. a pyrazolyl); (iv) contains two nitrogen atoms and one sulfur atom; (v) contains two nitrogen atoms and one oxygen atom; (vi) contains three nitrogen atoms.

In an embodiment, when Ring B is present (i.e. Z¹ represents (iii)), then such aromatic 6-membered ring may contain one nitrogen atom, so forming a pyridyl group (e.g. a 3-pyridyl group).

In an embodiment, further preferred compounds of the inventions include those in which:

none, but preferably, one or two (e.g. one) of R^{a}, R^{b}, R^{c}, R^{d} and R^{e} represents B¹ and the others represent hydrogen; and/or

one of R^{b}, R^{c} and R^{d} (preferably R) represents B¹ and the others represent hydrogen.

In a further embodiment, yet further preferred compounds of the inventions include those in which:

B¹ represents a substituent selected from:
(i) fluoro;
(ii) —OR^{e1};
(iii) C₁₋₃ alkyl, optionally substituted by one or more fluoro atom;
(iv) —C(O)N(R^{e2})R^{e3};
(v) —N(R^{e4})S(O)₂R^{e5};
(vi) —SF₅;

R^{e2} and R^{e4} independently represent hydrogen;

R^{e1}, R^{e3} and R^{e5} each independently represent C₁₋₃ alkyl (e.g. methyl) (e.g. optionally) substituted by one or more fluoro atoms.

In a further embodiment of the invention, B¹ represents a substituent selected from halo (e.g. fluoro), C₁₋₃ alkyl (optionally substituted by one or more fluoro atom) and —OR^{e1} (in which R^{e1} represents C₁₋₃ alkyl optionally substituted by one or more fluoro atom, so forming e.g. —OCF₃). In a specific embodiment, B¹ is selected from fluoro, —CH₃, —CF₃, —CH₂CF₃ and —OCF₃.

### Pharmacology

The compounds according to the invention have surprisingly been shown to be suitable for the treatment of a bacterial infection including a mycobacterial infection, particularly those diseases caused by pathogenic mycobacteria such as Mycobacterium tuberculosis (including the latent and drug resistant form thereof). The present invention thus also relates to compounds of the invention as defined hereinabove, for use as a medicine, in particular for use as a medicine for the treatment of a bacterial infection including a mycobacterial infection.

Such compounds of the invention may act by interfering with ATP synthase in M. tuberculosis, with the inhibition of cytochrome bd activity being the primary mode of action. Such bd inhibition may have an effect in killing mycobacteria (and hence having an anti-tuberculosis effect directly). However, as cytochrome bd is not necessarily essential for aerobic growth, it may have the most pronounced effect in combination with another inhibitor of a target of the electron transport chain of mycobacteria. Such compounds may be tested for cytochrome bd activity by testing in an enzymatic assay, and may also be tested for activity in the treatment of a bacterial infection (e.g. mycobacterial infection) by testing the kill kinetics, for example of such compounds alone or in combination (as mentioned herein, e.g. with one or more other inhibitor(s) of a (different) target of the electron transport chain of mycobacteria; such other different targets may be more implicated in aerobic growth).

Cytochrome bd is a component of the electron transport chain, and therefore may be implicated with ATP synthesis, for instance alone or especially with one or more other inhibitor(s) of a target of the electron transport chain of mycobacteria.

Further, the present invention also relates to the use of a compound of the invention, as well as any of the pharmaceutical compositions thereof as described hereinafter for the manufacture of a medicament for the treatment of a bacterial infection including a mycobacterial infection (for instance when such compound of the invention is used in combination with another inhibitor of a target of the electron transport chain of mycobacteria).

Accordingly, in another aspect, the invention provides a method of treating a patient suffering from, or at risk of, a bacterial infection, including a mycobacterial infection, which comprises administering to the patient a therapeutically effective amount of a compound or pharmaceutical composition according to the invention (for instance a therapeutically effective amount of a compound or pharmaceutical composition of the invention, in combination with one or more other inhibitor(s) of a target of the electron transport chain of mycobacteria).

The compounds of the present invention also show activity against resistant bacterial strains (for instance alone or in combination with another inhibitor of a target of the electron transport chain of mycobacteria).

Whenever used hereinbefore or hereinafter, that the compounds can treat a bacterial infection (alone or in combination) it is meant that the compounds can treat an infection with one or more bacterial strains.

The invention also relates to a composition comprising a pharmaceutically acceptable carrier and, as active ingredient, a therapeutically effective amount of a compound according to the invention. The compounds according to the invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, in particular, for administration orally or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations.

Depending on the mode of administration, the pharmaceutical composition will preferably comprise from 0.05 to 99% by weight, more preferably from 0.1 to 70% by weight, even more preferably from 0.1 to 50% by weight of the active ingredient(s), and, from 1 to 99.95% by weight, more preferably from 30 to 99.9% by weight, even more preferably from 50 to 99.9% by weight of a pharmaceutically acceptable carrier, all percentages being based on the total weight of the composition.

The pharmaceutical composition may additionally contain various other ingredients known in the art, for example, a lubricant, stabilising agent, buffering agent, emulsifying agent, viscosity-regulating agent, surfactant, preservative, flavouring or colorant.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof. The daily dosage of the compound according to the invention will, of course, vary with the compound employed, the mode of administration, the treatment desired and the mycobacterial disease indicated. However, in general, satisfactory results will be obtained when the compound according to the invention is administered at a daily dosage not exceeding 1 gram, e.g. in the range from 10 to 50 mg/kg body weight.

Given the fact that the compounds of the invention are useful against bacterial infections, the present compounds may be combined with other antibacterial agents in order to effectively combat bacterial infections. Where it is indicated that compounds may be useful against bacterial infections, we mean that those compounds may have activity as such or those compounds may be effective in combination (as described herein, e.g. with one or more other inhibitors of the electron transport chain of mycobacteria) by enhancing activity or providing synergistic combinations, for example as may be described in the experimental hereinafter.

Therefore, the present invention also relates to a combination of (a) a compound according to the invention, and (b) one or more other antibacterial agents (e.g. one or more other inhibitors of the electron transport chain of mycobacteria, for instance a cytochrome be inhibitor, an ATP synthase inhibitor, a NDH2 inhibitor and/or an inhibitor of the menaquinone synthesis pathway, such as a MenG inhibitor). The present invention also relates to such a compound or combination, for use as a medicine.

The present invention also relates to the use of a combination or pharmaceutical composition as defined directly above for the treatment of a bacterial infection.

A pharmaceutical composition comprising a pharmaceutically acceptable carrier and, as active ingredient, a therapeutically effective amount of (a) a compound according to the invention, and (b) one or more other antibacterial agents (e.g. one or more other inhibitors of the electron transport chain of mycobacteria, for instance a cytochrome be inhibitor, an ATP synthase inhibitor, a NDH2 inhibitor and/or an inhibitor of the menaquinone synthesis pathway, such as a MenG inhibitor), is also comprised by the present invention.

The weight ratio of (a) the compound according to the invention and (b) the other antibacterial agent(s) when given as a combination may be determined by the person skilled in the art. Said ratio and the exact dosage and frequency of administration depends on the particular compound according to the invention and the other antibacterial agent(s) used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention. A particular weight ratio for the present compound of the invention and another antibacterial agent may range from 1/10 to 10/1, more in particular from 1/5 to 5/1, even more in particular from 1/3 to 3/1.

The compounds according to the invention and the one or more other antibacterial agents may be combined in a single preparation or they may be formulated in separate preparations so that they can be administered simultaneously, separately or sequentially. Thus, the present invention also relates to a product containing (a) a compound according to the invention, and (b) one or more other antibacterial agents (e.g. one or more other inhibitors of the electron transport chain of mycobacteria, for instance a cytochrome be inhibitor, an ATP synthase inhibitor, a NDH2 inhibitor and/or an inhibitor of the menaquinone synthesis pathway, such as a MenG inhibitor), as a combined preparation for simultaneous, separate or sequential use in the treatment of a bacterial infection.

The other antibacterial agents which may be combined with the compounds of the invention are for example antibacterial agents known in the art. For example, the compounds of the invention may be combined with antibacterial agents known to interfere with the respiratory chain of Mycobacterium tuberculosis, including for example direct inhibitors of the ATP synthase (e.g. bedaquiline, bedaquiline fumarate or any other compounds that may have be disclosed in the prior art, e.g. compounds disclosed in WO2004/011436), inhibitors of ndh2 (e.g. clofazimine) and inhibitors of cytochrome bd. Additional mycobacterial agents which may be combined with the compounds of the invention are for example rifampicin (=rifampin); isoniazid; pyrazinamide; amikacin; ethionamide; ethambutol; streptomycin; para-aminosalicylic acid; cycloserine; capreomycin; kanamycin; thioacetazone; PA-824; delamanid; quinolones/fluoroquinolones such as for example moxifloxacin, gatifloxacin, ofloxacin, ciprofloxacin, sparfloxacin; macrolides such as for example clarithromycin, amoxycillin with clavulanic acid; rifamycins; rifabutin; rifapentin; as well as others, which are currently being developed (but may not yet be on the market; see e.g. http://www.newtbdrugs.org/pipeline.php). In particular, and as mentioned herein, compounds of the invention may be combined with one or more other inhibitors of the electron transport chain of mycobacteria, for instance a cytochrome be inhibitor, an ATP synthase inhibitor, a NDH2 inhibitor and/or an inhibitor of the menaquinone synthesis pathway, such as a MenG inhibitor. Given that the compounds of the invention might act as cytochrome bd inhibitors, and hence target the electron transport chain of the mycobacteria (thereby blocking energy production of mycobacteria), the compounds of the invention (cytochrome bd inhibitors), combinations with one or more other inhibitors of the electron transport chain is thought to be a potentially effective way of providing an efficient regimen against mycobacteria. Even if the compounds of the invention (cytochrome bd inhibitors) alone might not be effective against mycobacteria, combining with one or more other such inhibitors may provide an effective regimen where the activity of one or more components of the combination is/are enhanced and/or such combinations act more effectively (e.g. synergistically).

### General Preparation

The compounds according to the invention can generally be prepared by a succession of steps, each of which may be known to the skilled person or described herein.

### Experimental Part

Compounds of formula I may be prepared in accordance with the techniques employed in the examples hereinafter (and those methods know by those skilled in the art), for example by using the following techniques.

Compounds of formula (I) may be prepared by:

(i) conversion of a compound of formula (II),

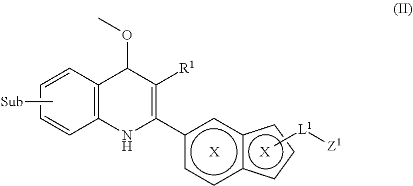

in which the integers are hereinbefore defined, by reaction with an appropriate reagent such as BBr₃ or NaSCH₃ (for example, as described in the examples);

(ii) reaction of a compound of formula (III),

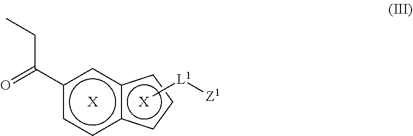

wherein the integers are as hereinbefore defined, with a compound of formula (IV),

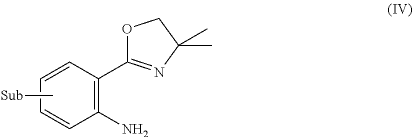

wherein the integers are hereinbefore defined, for example, in the presence of a reagent such as ZrCl₄, PTSA or the like, optionally in the presence of a solvent, such as an alcohol (e.g. butanol), under suitable reaction conditions (which may be further described in the examples).

It is evident that in the foregoing and in the following reactions, the reaction products may be isolated from the reaction medium and, if necessary, further purified according to methodologies generally known in the art, such as extraction, crystallization and chromatography. It is further evident that reaction products that exist in more than one enantiomeric form, may be isolated from their mixture by known techniques, in particular preparative chromatography, such as preparative HPLC, chiral chromatography. Individual diastereoisomers or individual enantiomers can also be obtained by Supercritical Fluid Chromatography (SCF).

The starting materials and the intermediates are compounds that are either commercially available or may be prepared according to conventional reaction procedures generally known in the art.

### EXPERIMENTAL

### Synthesis of Intermediate AA-2

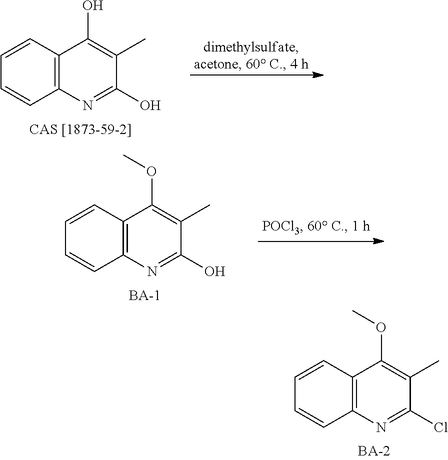

### Preparation of Intermediate BA-1

To a solution of 4-hydroxy-3-methylquinolin-2(1H)-one (CAS [1873-59-2], 2.00 g, 11.4 mmol) and K₂CO₃ (3.15 g, 22.8 mmol) in acetone (150 mL), was added dimethyl sulfate (1.30 mL, 13.7 mmol) at room temperature. The reaction mixture was stirred at 60° C. for 4 h then allowed to cool back to room temperature. The reaction mixture was concentrated to dryness, triturated with water and a white solid was recovered by filtration on a glass frit, washed with water, and vacuum-dried at 50° C. to afford intermediate BA-1 as a beige solid (1.76 g, 82%).

### Preparation of Intermediate BA-2

A mixture of BA-1 (3.08 g, 14.2 mmol) and POCl₃ (13.2 mL, 141 mmol) was stirred at 60° C. for 1 h, then allowed to cool back to room temperature and concentrated under reduced pressure.

The residue was carefully quenched with ice-water (300 mL) and extracted with DCM (3×100 mL). Combined organic phases were washed with a saturated aqueous solution of NaHCO₃ (100 mL), dried over sodium sulfate, filtered and concentrated under reduced pressure.

The residue (beige solid, 5 g) was purified by flash chromatography over silica gel (IR-50SI/200G column, cyclohexane/DCM 95:5 to 0:100, 45 min). The product fractions were collected, the solvent was evaporated, and the product was dried under high vacuum to afford intermediate BA-2 as a white solid, 2.38 g (81%).

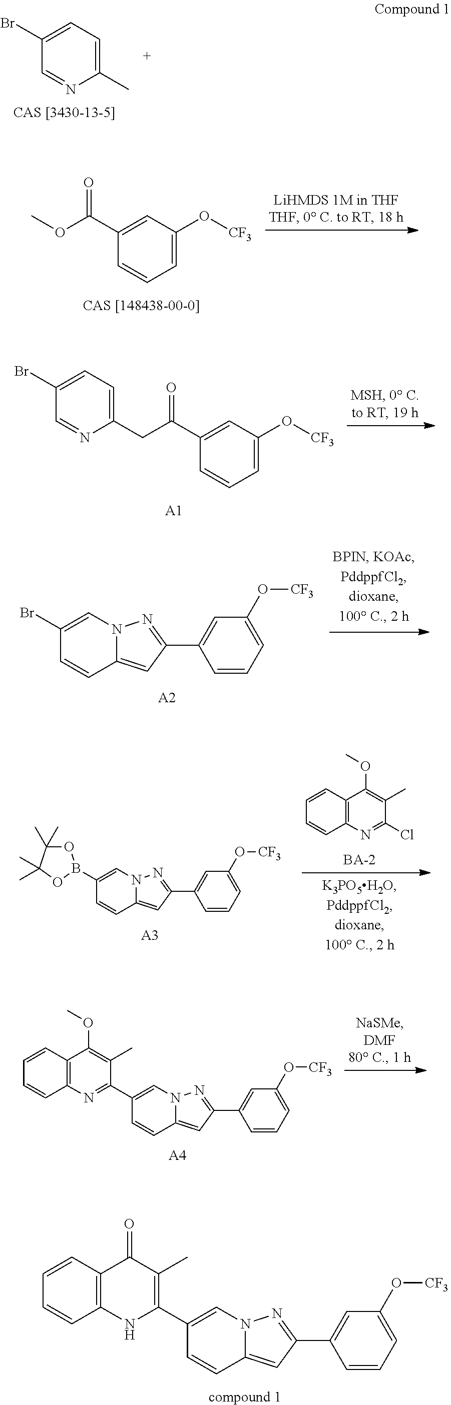

### Preparation of Intermediate A1

A mixture of 5-bromo-2-methyl-pyridine (CAS [3430-13-5], 840 mg, 4.88 mmol) and 3-Trifluoromethoxy-benzoic acid methyl ester (CAS [148438-00-0], 2.15 g, 9.77 mmol) in THE (2.2 mL) was cooled at 0° C. under nitrogen atmosphere. Then Lithium bis(trimethylsilyl)amide solution 1M in THE (CAS [4039-32-1], 14.7 mL, 14.7 mmol) was added dropwise at 0° C. and the reaction mixture was stirred at room temperature for 18 h. Ethyl acetate and water were added to the solution, then the organic phase was washed with water (10 mL) and brine (2×10 mL), dried over sodium sulfate, filtered and concentrated to dryness. The crude product was purified by flash chromatography on silica gel (IR-50SI/F0120, Cyclohexane/EtOAc from 100:0 to 90:10) to give intermediate A1 as a yellow solid 1.04 g (58%).

### Preparation of Intermediate A2

Crude solution of MSH (18.6 mL, max. 3.07 mmol) was cooled to 0° C., then a solution of intermediate A1 (745 mg, 2.07 mmol) in DCM (7.2 mL) was added dropwise at 0° C. The reaction mixture was allowed to warm to room temperature and stirred for 19 h. The reaction mixture was filtered then the filtrate was washed with water (25 mL), a saturated aqueous solution of NaHCO₃ (25 mL) and brine (2×25 mL). The organic layer was dried over Na₂SO₄, filtered and concentrated to dryness to afford a yellow oil. The crude residue was purified by flash chromatography over silica gel (IR-50SI/F0040, Cyclohexane/EtOAc from 100/0 to 95/5) to give intermediate A2 as a yellow solid, 0.29 g (39%).

Preparation of a fresh solution of (0-(mesitylsulfonyl)hydroxylamine) (=MSH) in CH₂Cl₂:

A solution of ethyl O-(2-mesitylenesulfonyl)acethydroxamate (CAS [38202-27-6], 1.22 g, 4.28 mmol) in 1,4-dioxane (9.8 mL) was stirred at 0° C. Perchloric acid (70%, 0.554 mL, 6.43 mmol) was added dropwise and the mixture was allowed to warm to room temperature and stirred for 15 min, then poured into ice water and stirred for 2 h. The precipitate was collected by filtration, washed with water and dissolved in CH₂Cl₂ (100 mL). The solution was dried over Na₂SO₄, filtered and concentrated to a volume of 26 mL. Considering quantitative yield, the freshly prepared crude solution of MSH in CH₂Cl₂ (with a concentration of max. 0.165 M) was used as such for amination. Concentrations of MSH solutions might vary in different experiments.

### Preparation of Intermediate A3

A nitrogen atmosphere purged mixture of intermediate A2 (287 mg, 0.804 mmol), bis(pinacolato)diboron (CAS [73183-34-3), 245 mg, 0.964 mmol), potassium acetate (197 mg, 2.01 mmol) and [1,1′-bis(diphenylphosphino)ferrocene] dichloropalladium (58.8 mg, 0.080 mmol) in 1,4-dioxane (2.5 mL) was stirred at 100° C. for 2 h. The reaction mixture was filtered through Celite®, the filter cake was rinsed with EtOAc (20 mL) and the filtrate was concentrated to dryness to afford intermediate A3 as a black oil, 0.53 g (purity 60%, quantitative). The product was used in the next step.

### Preparation of Intermediate A4

A mixture of BA-2 (111 mg, 0.536 mmol), intermediate A3 (533 mg, max. 0.804 mmol) and potassium phosphate monohydrate (370 mg, 1.61 mmol) in a mixture of 1,4-dioxane (0.8 mL) and water (0.2 mL) was purged with argon, then [1,1′-bis(diphenylphosphino)ferrocene] dichloropalladium (39.2 mg, 0.054 mmol) was then added and the mixture was purged again with argon and stirred at 100° C. for 2 h. The reaction mixture was filtered through Celite®, the filter cake was rinsed with EtOAc (20 mL) and the filtrate was concentrated to dryness to afford a black oil. The crude oil was purified by flash chromatography over silica gel (IR-50SI/F0025, Cyclohexane/EtOAc from 100/0 to 80/20) to give intermediate A4 as a yellow solid, 0.22 g, 90%.

### Preparation of Compound 1

To a solution of intermediate A4 (217 mg, 0.483 mmol) in DMF (2.1 mL) was added sodium thiometoxyde (169 mg, 2.41 mmol) at room temperature and the resulting mixture was stirred at 80° C. for 1 h. The reaction mixture was quenched with water (10 mL), the aqueous layer was filtered on a glass frit and the collected solid was washed with water to give after vacuum-drying a yellow solid, 0.28 g. It was purified by flash chromatography over silica gel (IR-50SI/F0025, DCM/MeOH from 100/0 to 96/4) to give as a yellow solid, 0.13 g.

The solid was triturated with a mixture of DCM and MeOH 90/10, the mixture was concentrated to dryness to give a yellow solid. Then it was dissolved in refluxing EtOAc (80 mL, stirred for 15 min) and concentrated to dryness to give a white solid, dried under high vacuum at 60° C. to give Compound 1 as a white solid, 0.118 g (56%).

mp: 243.7° C. (DSC 1 Mettler Toledo 5° C./min).

1H NMR (400 MHz, DMSO-d6) δ ppm 11.71 (s, 1H), 9.11 (s, 1H), 8.15 (d, J=8.2 Hz, 1H), 8.09 (d, J=8.0 Hz, 1H), 7.99 (s, 1H), 7.89 (d, J=9.1 Hz, 1H), 7.68-7.57 (m, 3H), 7.45-7.40 (m, 2H), 7.35-7.30 (m, 2H), 2.00 (s, 3H).

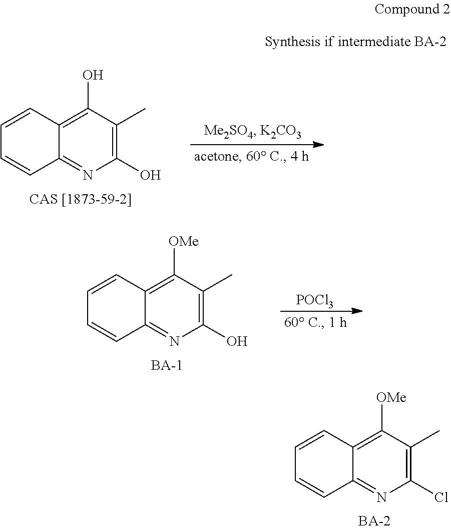

### Preparation of Intermediate BA-1

To a solution of 4-hydroxy-3-methylquinolin-2(1H)-one (CAS [1873-59-2], 2.00 g, 11.4 mmol) and K₂CO₃ (3.15 g, 22.8 mmol) in acetone (150 mL), was added dimethyl sulfate (1.30 mL, 13.7 mmol) at room temperature. The reaction mixture was stirred at 60° C. for 4 h then allowed to cool back to room temperature. The reaction mixture was concentrated to dryness, triturated with water and a white solid was recovered by filtration on a glass frit, washed with water, and vacuum-dried at 50° C. to afford intermediate BA-I as a beige solid (1.76 g, 82%).

### Preparation of Intermediate BA-2

A mixture of intermediate BA-1 (1.45 g, 7.66 mmol) and POCl₃ (7.14 mL, 76.6 mmol) was stirred at 60° C. for 1 h. The resulting mixture was allowed to cool back to room temperature, combined with another reaction mixture obtained from 0.264 mmol of intermediate ZZ-1 and concentrated to dryness. The residue was carefully quenched with ice-water and extracted with CH₂Cl₂. The combined organic layers were washed with a saturated aqueous solution of NaHCO₃, dried over Na₂SO₄ and concentrated to dryness. The crude residue was purified by flash chromatography over silica gel (IR50SI, cyclohexane/CH₂Cl₂ from 95:5 to 40:60) to afford intermediate BA-2 as a white solid (1.34 g, 81%).

### Synthesis of Intermediate BB-2

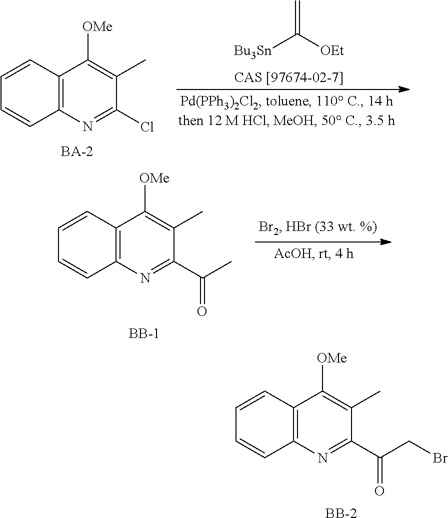

### Preparation of Intermediate BB-1

A solution of intermediate BA-2 (3.00 g, 14.4 mmol) and tributyl(1-ethoxyvinyl)tin (CAS [97674-02-7], 6.35 mL, 18.8 mmol) in toluene (60 mL) was argon-purged bis(triphenylphosphine)palladium(II) dichloride (0.507 g, 0.722 mmol) was added and the mixture was purged again with argon and stirred at 110° C. for 14 h. The reaction mixture was concentrated under reduced pressure to approximately 15 mL, then MeOH (60 mL) and a 12 M aqueous solution of HCl (15 mL) were added and the mixture was stirred at 50° C. for 3.5 h. MeOH was removed under reduced pressure and 3 M aqueous NaOH was added until pH˜ 7. The aqueous layer was extracted with CH₂Cl₂ and the combined organic layers were dried over Na₂SO₄ and concentrated to dryness. The residue was purified by flash chromatography over silica gel (IR50SI, cyclohexane/EtOAc 95:5) to afford intermediate BB-1 as a white solid (2.09 g, 64%).

### Preparation of Compound BB-2

To a solution of intermediate intermediate BB-1 (2.09 g, 9.20 mmol) in AcOH (40 mL) were added successively HBr 33 wt. % in acetic acid (6.50 mL, 37.1 mmol) and bromine (0.498 mL, 9.66 mmol) and the mixture was stirred at room temperature for 4 h. The reaction mixture was concentrated to dryness, then the residue was taken up with CH₂Cl₂ and a saturated aqueous solution of NaHCO₃ and the aqueous layer was extracted with CH₂Cl₂. The combined organic layers were dried over sodium sulfate, filtered and concentrated to dryness. The crude product intermediate BB-2 was considered as quantitative and used as such in the next step (2.84 g containing maximum 9.20 mmol).

### Synthesis of Intermediate BC-1

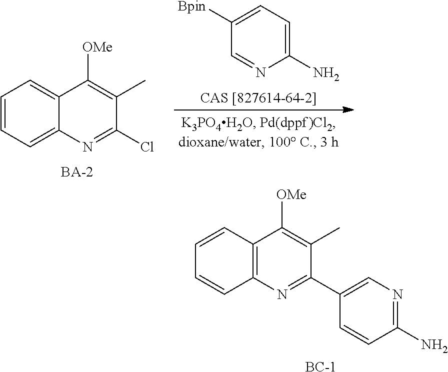

### Preparation of Compound BC-1

An argon-purged mixture of intermediate BA-2 (1.00 g, 4.82 mmol), 2-aminopyridine-5-boronic acid pinacol ester (CAS [827614-64-2], 1.27 g, 5.78 mmol), K₃PO₄.H₂O (3.33 g, 14.4 mmol) and Pd(dppf)Cl₂ (0.352 g, 0.482 mmol) in a mixture of 1,4-dioxane (7 mL) and water (1.75 mL) was stirred at 100° C. for 3 h, then allowed to cool back to room temperature. The reaction mixture was filtered on a pad of Celite® which was rinsed with EtOAc and the filtrate was washed with water, brine, dried over Na₂SO₄ and concentrated to dryness. The crude residue was purified by flash chromatography over silica gel (IR50SI, CH₂Cl₂/MeOH from 100:0 to 94:6) to afford intermediate BC-1 as a brown solid (1.14 g, 89%).

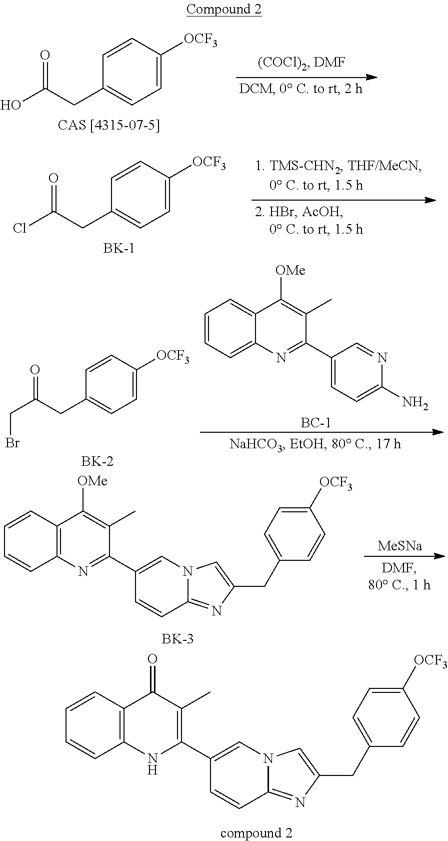

### Preparation of Intermediate BK-1

To a solution of 4-(trifluoromethoxy)phenylacetic acid (CAS [4315-07-5], 2.00 g, 9.09 mmol) and N,N-dimethylformamide (0.0352 mL, 0.454 mmol) in CH₂Cl₂ (15 mL) was added oxalyl chloride (0.846 mL, 9.99 mmol) dropwise at 0° C. under argon atmosphere. The reaction mixture was stirred at room temperature for 2 h then concentrated to dryness to afford intermediate BK-1 as a yellow liquid (2.09 g, 96%).

### Preparation of Intermediate BK-2

To a solution of intermediate BK-1 (2.09 g, 8.76 mmol) in a mixture of THF (5 mL) and MeCN (5 mL) was added (trimethylsilyl)diazomethane (2 M in Et₂O) (8.76 mL, 17.5 mmol) dropwise at 0° C. under argon atmosphere. The reaction mixture was stirred at room temperature for 1.5 h then concentrated to dryness under reduced pressure. The crude mixture was dissolved in AcOH (35 mL), cooled to 0° C. and 48 wt. % aqueous HBr (1.67 ml, 14.7 mmol) was added dropwise. The reaction mixture was stirred at room temperature for 1.5 h, quenched with a 3M NaOH aqueous solution until pH-7. The aqueous layer was extracted with EtOAc. The combined organic layers were dried over Na₂SO₄, filtered and concentrated to dryness. The crude residue was purified by flash chromatography over silica gel (IR50SI, cyclohexane/CH₂Cl₂ from 100:0 to 70:30) to afford intermediate BK-2 as a brown liquid (1.58 g, 59%).

### Preparation of Intermediate BK-3

A mixture of intermediate BC-1 (0.250 g, 0.942 mmol), intermediate BK-2 (0.509 g, 0.942 mmol) and NaHCO₃ (0.158 g, 1.89 mmol) in EtOH (9 mL) was stirred at 80° C. for 17 h, then allowed to cool back to room temperature. The reaction mixture was then concentrated to dryness and the residue was taken in CH₂Cl₂ and washed with water. The aqueous layer was then extracted with CH₂Cl₂ and the combined organic layers were dried over Na₂SO₄, filtered and concentrated to dryness. The residue was purified by flash chromatography over silica gel (IR50SI, CH₂Cl₂/MeOH from 100:0 to 96:4) to afford intermediate BK-3 as a brown foam (0.293 g, 67%).

### Preparation of Compound 2

A mixture of intermediate BK-3 (0.266 g, 0.574 mmol) and NaSMe (0.201 g, 2.87 mmol) in DMF (1.5 mL) was stirred at 80° C. for 1 h then allowed to cool back to room temperature. The reaction mixture was then diluted with isopropyl acetate and washed with water, a saturated aqueous solution of NH₄Cl and brine. The aqueous layer was then extracted with CH₂Cl₂. The combined organic layers were dried over Na₂SO₄, filtered and concentrated to dryness. The crude residue was purified by flash chromatography over silica gel (IR50SI, CH₂Cl₂/MeOH from 100:0 to 95:5) to afford compound 2 as a beige solid (0.174 g, 67%).

1H NMR (400 MHz DMSO-d₆) δ ppm 11.64 (s, 1H), 8.84 (dd, J=1.7, 0.9 Hz, 1H), 8.13 (dd, J=8.2, 1.3 Hz, 1H), 7.81 (s, 1H), 7.66-7.60 (m, 2H), 7.57 (d, J=8.3 Hz, 1H), 7.45 (d, J=8.7 Hz, 2H), 7.37 (dd, J=9.2, 1.8 Hz, 1H), 7.34-7.28 (m, 3H), 4.13 (s, 2H), 1.95 (s, 3H).

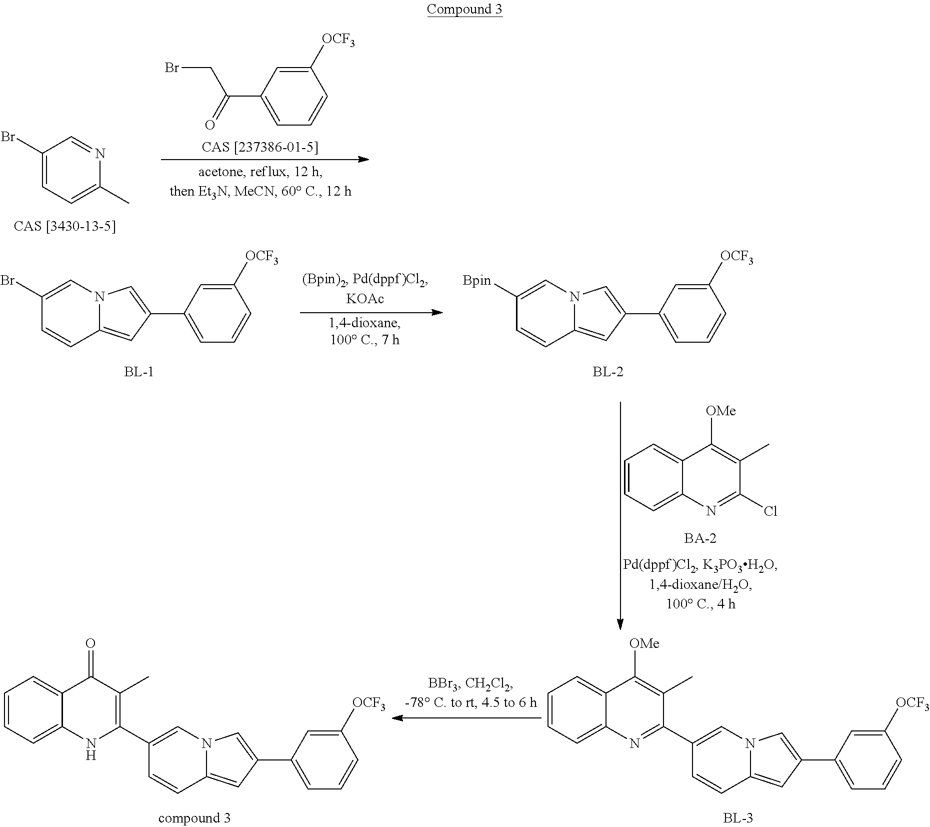

### Preparation of Intermediate BL-1

A mixture of bromo-1-[3-(trifluoromethoxy)phenyl]ethan-1-one (CAS [237386-01-5], 2.00 g, 7.07 mmol) and 5-bromo-2-methylpyridine (CAS [3430-13-5], 1.58 g, 9.19 mmol) in acetone (20 ml) was stirred at 90° C. for 12 h. The formed solid was collected by filtration and diluted in Et₃N (4.92 ml, 35.3 mmol) and MeCN (40 ml). The resulting yellow solution was stirred at 60° C. for 12 h and then concentrated to dryness. The residue was dissolved in dichloromethane, washed with saturated aqueous NaHCO₃, dried over Na₂SO₄, filtered and concentrated to dryness to afford intermediate BL-1 as yellow solid (0.882 g, 35%). The product was used as such for the next step.

### Preparation of Intermediate BL-2 and BL-3

An argon-purged mixture of intermediate BL-1 (441 mg, 1.24 mmol), bis(pinacolato)diboron (377 mg, 1.49 mmol), KOAc (304 mg, 3.10 mmol) and Pd(dppf)Cl₂ (90.6 mg, 0.124 mmol) in 1,4-dioxane (18 ml) was stirred at 100° C. for 7 h. The mixture was filtered through Celite®, the filter cake was rinsed with EtOAc and the filtrate was concentrated to dryness to afford crude intermediate BL-2 (840 mg) as a black oil. A mixture of this crude intermediate BL-2 (840 mg) with intermediate BA-2 (110 mg, 0.531 mmol) and K3PO4.H2O (367 mg, 1.59 mmol) in 1,4-dioxane (18 ml) and water (1 ml) was argon-purged, then Pd(dppf)Cl2 (38.9 mg, 0.053 mmol) was added, the mixture was purged again with argon and stirred at 100° C. for 4 h. The reaction mixture was filtered through a pad of Celite® which was rinsed with EtOAc and the filtrate was washed with water and brine, dried over Na2SO4, filtered and concentrated to dryness. The crude product was purified by flash chromatography over silica gel (IR-50SI, cyclohexane/EtOAc gradient from 100/0 to 90/10) to give intermediate BL-3 as a yellowish solid (82.1 mg).

### Preparation of Compound 3

A 1 M solution of BBr3 in CH2Cl2 (0.892 ml, 0.892 mmol) was added dropwise to a solution of intermediate BL-3 (80.0 mg, 0.178 mmol) in CH2Cl2 (3.8 ml) cooled to −78° C. under argon atmosphere, and the resulting mixture was allowed to warm back to room temperature and stirred for 4.5 h, then quenched with water and extracted with CH2Cl2. The combined organic layers were washed with brine, dried over Na2SO4, filtered and concentrated to dryness. The residue was combined with additional compound obtained analogously in a second experiment (reaction time 6 h) starting from 75.1 mg (0.167 mmol) BL-3 and purified by reversed phase flash chromatography (IR-50C18; water/MeCN from 70/30 to 0/100), triturated in MeOH and vacuum-dried (50° C.) to afford compound 3 as a yellow solid (22.3 mg, combined yield: 15%).

1H NMR (400 MHz, DMSO-d6) □ ppm 11.66 (s, 1H), 8.57 (s, 1H), 8.21 (d, J=1.3 Hz, 1H), 8.13 (dd, J=8.1 Hz, 1 Hz, 1H), 7.82-7.78 (m, 1H), 7.72 (s, 1H), 7.66-7.53 (m, 4H), 7.33-7.29 (m, 1H), 7.26 (d, J=8.4 Hz, 1H), 7.02 (s, 1H), 6.89 (dd, J=9.1 Hz, 1.5 Hz, 1H), 2.00 (s, 3H).

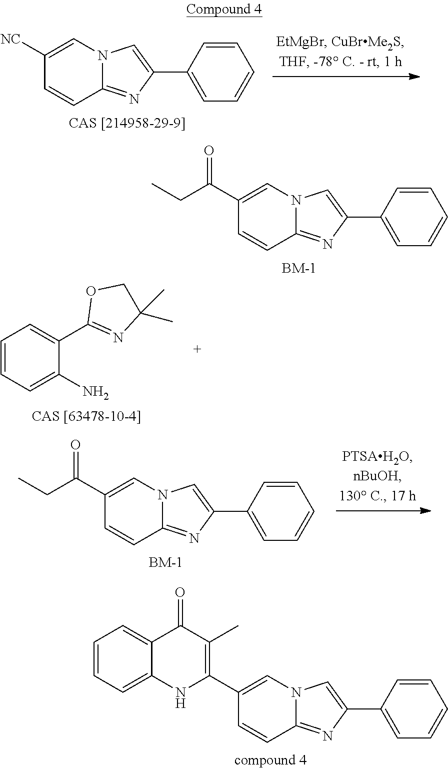 [SMILES: N#Cc1ccc2nc(-c3ccccc3)cn2c1 ; CCC(=O)c1ccc2nc(-c3ccccc3)cn2c1 ; CC1(C)COC(c2ccccc2N)=N1 ; CCC(=O)c1ccc2nc(-c3ccccc3)cn2c1 ; Cc1c(-c2ccc3nc(-c4ccccc4)cn3c2)[nH]c2ccccc2c1=O]

### Preparation of Intermediate BM-1

Under nitrogen atmosphere, to suspension of 2-Phenylimidazo[1,2-a]pyridine-6-carbonitrile (CAS [214958-29-9], 537 mg, 2.45 mmol) in dry THF (10.7 mL) at −78° C., were added CuBre.SMe2 (20.1 mg, 0.098 mmol) and a 1M solution of ethylmagnesium bromide in THF (3.67 mL, 3.67 mmol). The reaction mixture was then warmed up to room temperature and stirred for 1 h. The reaction mixture was hydrolyzed with water for 1 h. The aqueous phase was extracted with CH2Cl2. The organic phase was washed with brine, dried over MgSO4, filtered and evaporated to dryness to give a brown solid which was purified by flash chromatography over silica gel (irregular SiOH, 15-40 μm, 40 g, Grace, dry loading (silica), mobile phase gradient: from DCM 100% to DCM 90%, MeOH 10% over 15 CV) to give intermediate BM-1 as an orange solid (0.360 g, 59%).

### Preparation of Compound 4

A solution of intermediate BM-1 (209 mg, 1.10 mmol), 2-(2-Aminophenyl)-4,4-dimethyl-2-oxazoline (CAS [63478-10-4], 302 mg, 1.21 mmol) and p-toluenesulfonic acid monohydrate (83 mg, 0.44 mmol) in n-butanol (3 mL) was stirred at 130° C. under nitrogen overnight. The reaction mixture was evaporated to dryness. The residue was taken up in EtOAc and washed with water. The organic phase was washed with brine, dried over MgSO4, filtered and evaporated to dryness to give a residue. The residue was purified by flash chromatography over silica gel (irregular SiOH, 15-40 μm, 40 g, Grace, dry loading (silica), mobile phase gradient: from DCM 100% to DCM 90%, MeOH 10% over 15 CV) to give a yellow solid.

The solid was combined with another batch, triturated in Et2O, the supernatant was removed with a pipette to give an off-white solid, which was dissolved in EtOH and slowly concentrated to dryness under vacuum to give compound 4 as off-white solid (0.124 g, combined yield 16%).

1H NMR (500 MHz, DMSO-d6) □ ppm 11.74 (br s, 1H) 8.90 (s, 1H) 8.52 (s, 1H) 8.15 (d, J=8.0 Hz, 1H) 8.03 (d, J=7.3 Hz, 2H) 7.77 (d, J=9.1 Hz, 1H) 7.65 (t, J=7.7 Hz, 1H) 7.60 (d, J=8.2 Hz, 1H) 7.42-7.51 (m, 3H) 7.30-7.39 (m, 2H) 1.99 (s, 3H).

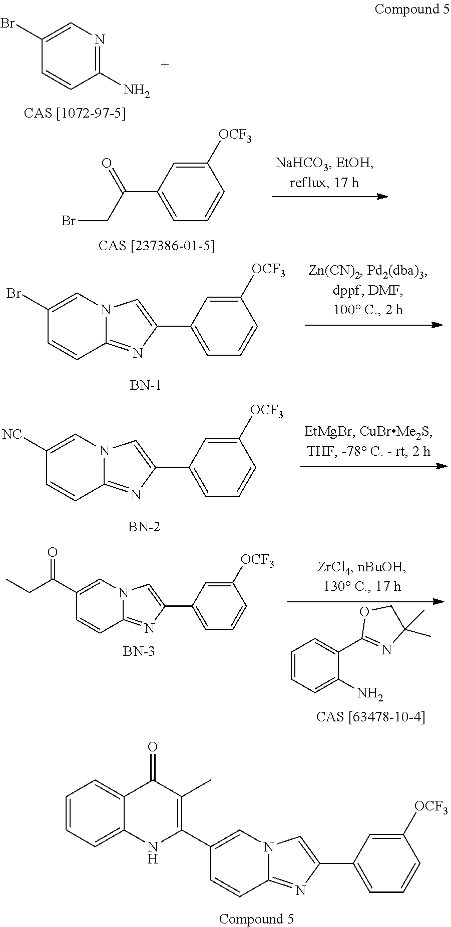

### Preparation of Intermediate BN-1

A mixture of 5-bromo-2-aminopyridine (CAS [1072-97-5], 1.8 g, 10.4 mmol), 2-Bromo-1-(3-trifluoromethoxyphenyl)ethanone (CAS [237386-01-5], 3 g, 10.6 mmol) and NaHCO3(1.2 g, 14.3 mmol) in ethanol (14 mL) was refluxed for 17 h. After cooling down to room temperature, water was added and the solid was filtered. The precipitate was washed with water and ethanol then dried under vacuum to give intermediate BN-1 as a light brown solid (3.22 g, 87%).

### Preparation of Intermediate BN-2

In a sealed tube, a mixture of dppf (160 mg, 0.289 mmol) in dry DMF (14 mL) was purged with nitrogen for 2 minutes, then Pd₂(dba)₃ (130 mg, 0.142 mmol), Zn(CN)₂ (200 mg, 1.70 mmol) and intermediate BN-1 (1 g, 2.8 mmol) were added and the mixture was heated at 100° C. for 2 h. The mixture was filtered off and the cake was washed with EtOAc. The filtrate was evaporated in vacuo to afford a solid which was purified by flash chromatography over silica gel (irregular SiOH, 15-40 μm, 50 g, Merck, dry loading (Celite®), mobile phase gradient: from heptane 90%, EtOAc 10% to Heptane 55%, EtOAc 45% over 12CV) to afford intermediate BN-2 as a yellow solid (0.795 g, 94%).

### Preparation of Intermediate BN-3

In a sealed tube, CuBreSMe₂ (40 mg, 0.195 mmol) and B (3.2 mL, 3.2 mmol) were added to a suspension of intermediate BN-2 (745 mg, 2.46 mmol) in dry Me-THF (7.5 mL) under nitrogen at −78° C. The reaction mixture was then warmed up to room temperature and stirred for 2 h. Aqueous 1M HCl was added and the mixture was stirred at room temperature for 15 minutes. Water and CH₂Cl₂ were added, the layers were separated and the aqueous layer was extracted with CH₂Cl₂ (twice). The combined organic layers were dried over MgSO₄, filtered off and evaporated in vacuo to afford a solid which was purified by flash chromatography over silica gel (irregular SiOH, 15-40 μm, 50 g, Merck, dry loading (Celite®), mobile phase gradient: from Heptane 85%, EtOAc 15% to Heptane 50%, EtOAc 50% over 12 CV) to give intermediate BN-3 as a yellow solid (0.531 g, 65%).

### Preparation of Compound 5

A solution of intermediate BN-3 (225 mg, 0.673 mmol), 2-(2-Aminophenyl)-4,4-dimethyl-2-oxazoline (CAS [63478-10-4], 155 mg, 0.815 mmol) and zirconium (IV) chloride (60 mg, 0.257 mmol) in n-butanol (3 mL) was stirred at 130° C. under nitrogen overnight. The reaction mixture was combined with others batches and filtered through a pad of Celite®. The filtrate was evaporated in vacuo. The residual gum was solubilized in EtOAc then washed with water and brine. The organic layer was dried over MgSO4, filtered off and evaporated in vacuo to give a brown gum. The gum was purified by flash chromatography over silica gel (irregular SiOH, 15-40 μm, 50 g, Merck, dry loading (Celite®), mobile phase gradient: from heptane 100% to heptane 25%, EtOAc 75% over 12 CV) to afford a pale yellow gum which was triturated and sonicated in Et2O/DCM (9:1), filtered over a glass-frit then dried under high vacuum (50° C., 16 h) to give an off-white solid. The solid was co-evaporated with EtOH (twice), triturated in iPr₂O then dried under high vacuum to give compound 5 a white solid (0.103 g, combined yield 14%).

1H NMR (500 MHz, DMSO-d₆) δ ppm 11.75 (br s, 1H) 8.92 (s, 1H) 8.65 (s, 1H) 8.15 (br d, J=8.2 Hz, 1H) 8.06 (br d, J=7.9 Hz, 1H) 8.00 (br s, 1H) 7.80 (d, J=9.1 Hz, 1H) 7.57-7.68 (m, 3H) 7.48 (br d, J=9.5 Hz, 1H) 7.30-7.38 (m, 2H) 1.98 (s, 3H)

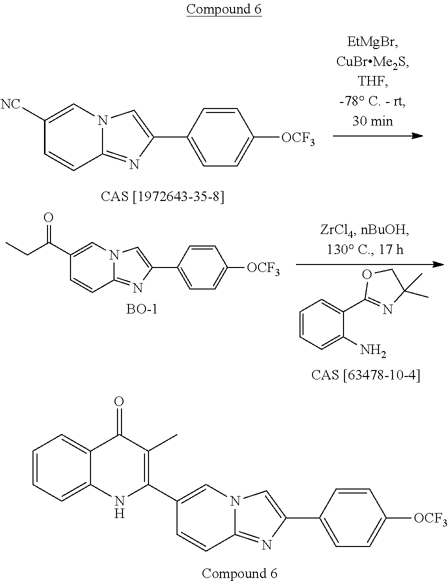

### Preparation of Intermediate BO-1

Accordingly, intermediate BO-1 was prepared in the same way as intermediate BN-3 starting from 2-[4-(trifluoromethoxy)phenyl]-imidazo[1,2-a]pyridine-6-carbonitrile, (CAS [1972643-35-8], 770 mg, 2.54 mmol) yielding 0.394 g, yield: 46%.

### Preparation of Compound 6

Accordingly, compound 6 was prepared in the same way as compound 5 starting from intermediate BO-1 (394 mg, 1.18 mmol) and 2-(2-Aminophenyl)-4,4-dimethyl-2-oxazoline (CAS [63478-10-4], 270 mg, 1.42 mmol) yielding 0.103 g, yield: 20%.

1H NMR (500 MHz, DMSO-d₆) δ ppm 11.73 (s, 1H) 8.92 (s, 1H) 8.57 (s, 1H) 8.15 (d, J=8.5 Hz, 3H) 7.78 (d, J=9.1 Hz, 1H) 7.65 (t, J=7.7 Hz, 1H) 7.59 (d, J=8.1 Hz, 1 H) 7.44-7.50 (m, 3H) 7.33 (t, J=7.5 Hz, 1H) 1.99 (s, 3H)

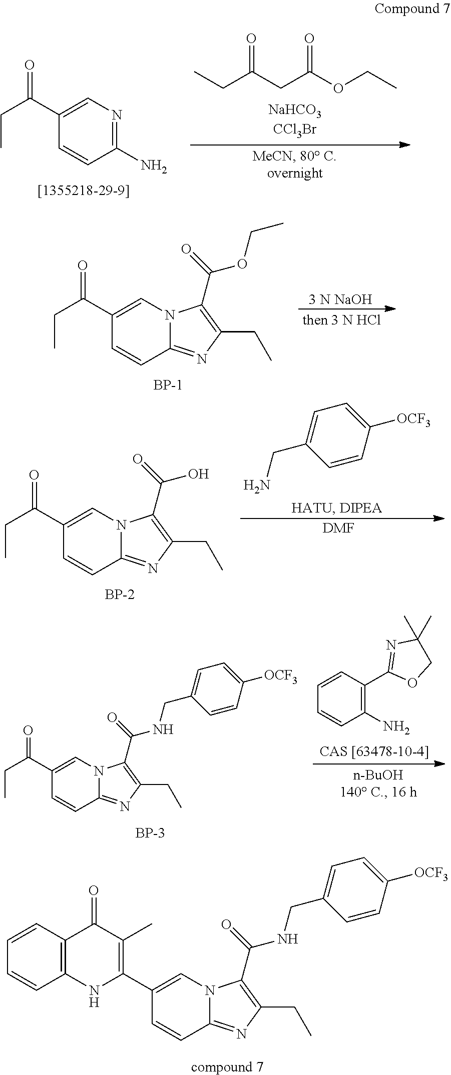

### Preparation of Intermediate BP-1

A sealed tube was loaded with 1-(6-aminopyridin-3-yl)propan-1-one [1355218-29-9](50 mg, 0.33 mmol), NaHCO₃ (56 mg, 0.67 mmol), acetonitrile (0.59 mL), ethyl-3-oxovalerate (0.048 mL, 0.330 mmol), and bromotrichloromethane (0.066 mL, 0.670 mmol). The tube was sealed and the reaction mixture was heated at 80° C. overnight. The reaction mixture was diluted with water (5 mL) and the aqueous layer was extracted with EtOAc (5×15 mL). Combined organic layers were washed with brine, dried (MgSO4) and solvent was removed in vacuo. Purification by flash column chromatography on silica gel (GraceResolv, 4 g, SiOH 25-40 μM, dry loaded on Celite®, heptane:ethyl acetate; 40:60) yielded intermediate BP-1 as an offwhite solid, 24.6 mg, 27% yield.

### Preparation of Intermediate BP-2

To a solution of intermediate BP-1 (44.3 mg, 0.16 mmol) in ethanol (3 mL) was added NaOH (3N solution, 0.16 mL, 0.48 mmol) at rt. The reaction mixture was stirred at room temperature until completion, quenched with HCl (3 N solution, 0.16 mL, 0.48 mmol) and solvent was removed in vacuo. The crude product intermediate BP-2 (39.8 mg, 0.16 mmol) was used in the next step without purification.

### Preparation of Intermediate BP-3

To a solution of intermediate BP-2 (39.8 mg, 0.16 mmol) and HATU (67.5 mg, 0.18 mmol) in DMF (3 mL) was added DIPEA (0.083 mL, 0.48 mmol). The reaction mixture was stirred at room temperature for 30 min prior to add 4-(trifluoromethoxy)benzylamine (0.027 mL, 0.18 mmol). The reaction mixture was stirred overnight and diluted with EtOAc (20 mL). The organic layer was washed with brine (5×10 mL), dried (MgSO4) and solvent was removed in vacuo. Purification by flash column chromatography on silica gel (GraceResolv, 4 g, SiOH 25-40 μM, dry loaded on Celite®, heptane:ethyl acetate; 60:40) yielded intermediate BP-3 as a brow solid, 62.8 g, 93% yield (contaminated with HATU derivatives and used as such in the next step).

### Preparation of Compound 7

To a solution of 2-(2-Aminophenyl)-4,4-dimethyl-2-oxazoline (CAS [63478-10-4], 25.9 mg, 0.14 mmol) and intermediate BP-3 (62.8 mg, 0.15 mmol) in n-butanol (0.32 mL) was added PTSA (14.1 mg, 0.082 mmol) at room temperature. The reaction mixture was heated for 16 h at 140° C. Purification by flash column chromatography on silica gel (GraceResolv, SiOH 25-40 μM, dry loaded on Celite®, CH₂Cl₂:MeOH:AcOH; 95:5:0.1) and subsequent trituration (MeCN:EtOH; 8:2) yielded compound 7 as a colourless solid, 28 mg, 33% (over 3 steps.

¹H NMR (500 MHz, DMSO-d₆) δ ppm 11.72 (br s, 1H), 9.21 (s, 1H), 8.56 (t, J=5.8 Hz, 1H), 8.14 (dd, J=8.0, 1.1 Hz, 1H), 7.79 (d, J=9.1 Hz, 1H), 7.62-7.67 (m, 1H), 7.56-7.61 (m, 2H), 7.51 (d, J=8.8 Hz, 2H), 7.30-7.36 (m, 3H), 4.56 (d, J=5.7 Hz, 2 H), 3.06 (q, J=7.5 Hz, 2H), 1.95 (s, 3H), 1.31 (t, J=7.6 Hz, 3H); ¹⁹F NMR (471 MHz, DMSO-d₆) δ ppm −56.84 (s, 1 F).

The following compounds are/were also prepared in accordance with the methods described herein:

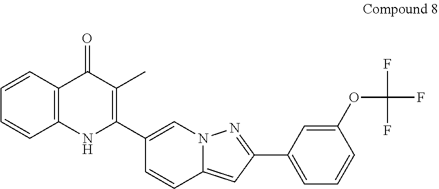 [SMILES: Cc1c(-c2ccc3cc(-c4cccc(OC(F)(F)F)c4)nn3c2)[nH]c2ccccc2c1=O]

### Abbreviations

K₂CO₃ Potassium carbonate

CH₂Cl₂ Dichloromethane

DCM Dichloromethane

Na₂SO₄ Sodium Sulfate

NaHCO₃ Sodium Bicarbonae

THE Tetrahydrofuran

EtOAc Ethyl Acetate

MSH O-Mesitylenesulfonylhydroxylamine

MeOH Methanol

Et₂O Diethyl Ether

NH₄Cl Ammonium Chloride

EtOH Ethanol

H₂ Dihydrogen gas

POCl₃ Phosphoryl chloride

LiHMDS Lithium bis(trimethylsilyl)amide

DMF Dimethylformamide

Analytical Methods

Reactions

Were in general carried out in anhydrous solvents under argon atmosphere if no other gas atmosphere was required.

NMR

Was carried out on a Bruker 400 MHz spectrometer.

Melting Points

Were determined by DSC on a Mettler-Toledo DSC1 instrument (using aluminum standard 40 μL pans with air as purge gas and a thermal gradient between −10° C. and 350° C.) or on a melting point apparatus Buchi M-560, both applying indicated heating rates.

For flash chromatography, in general the following stationary phases (Interchim) were used:

Silica gel IR-50SI (irregular, 50 μm), silica gel PF-15SIHP (spherical, 15 μm) or C18-reversed silica gel IR-50C18 (irregular, 50 μm).

LCMS

The mass of some compounds was recorded with LCMS (liquid chromatography mass spectrometry). The methods used are described below.

General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time, etc) in order to obtain ions allowing the identification of the compound's nominal monoisotopic molecular weight (MW). Data acquisition was performed with appropriate software. Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc). For molecules with multiple isotopic patterns (Br, Cl . . . ), the reported value is the one obtained for the lowest isotope mass. All results were obtained with experimental uncertainties that are commonly associated with the method used.

Hereinafter, the following abbreviations may be used: “SQD” means Single Quadrupole Detector, “RT” room temperature, “BEH” bridged ethylsiloxane/silica hybrid, “HSS” High Strength Silica, “DAD” Diode Array Detector, “MSD” Mass Selective Detector.

TABLE
LCMS Method codes (Flow expressed in mL/min; column temperature (T)
in ° C.; Run time in minutes).

| Method | | | | | Flow | Run |
|---|---|---|---|---|---|---|
| Code | Instrument | Column | Mobile phase | gradient | Column T | time |
| Method | Thermo- | Agilent: | A: HCOOH | 98% A for 3 | 1 | 28 |
| A | scientific | Eclipse | 0.1% in | min, to 0% A | 30 | |
| | Ultimate | XDB C18 | water/ | in 12 min, held | | |
| | 3000 | (5 μm, | B: | for 5 min, back | | |
| | DAD and | 4.6 × 150 | HCOOH | to 98% A in 2 | | |
| | Brucker | mm) | 0.05% in | min, held for 6 | | |
| | HCT | | CH3CN | min | | |
| | ultra | | | | | |
| Method | Thermo- | Agilent: | A: HCOOH | 98% A for 2 | 1 | 18.4 |
| B | scientific | Poroshell | 0.1% in | min, to 0% A | 30 | |
| | Ultimate | EC-C18 | water/ | in 10 min, held | | |
| | 3000 | (4 μm, | B: | for 3.4 min, | | |
| | DAD and | 4.6 × 100 | HCOOH | back to 98% A | | |
| | Brucker | mm) | 0.05% in | in 1.3 min, held | | |
| | HCT | | CH3CN | for 1.7 min | | |
| | ultra | | | | | |
| Method | Thermo- | Agilent: | A: HCOOH | 50% A for 2 | 1 | 18.4 |
| C | scientific | Poroshell | 0.1% in | min, to 0% A | 30 | |
| | Ultimate | EC-C18 | water/ | in 10 min, held | | |
| | 3000 | (4 μm, | B: | for 3.4 min, | | |
| | DAD and | 4.6 × 100 | HCOOH | back to 50% A | | |
| | Brucker | mm) | 0.05% in | in 1.3 min, held | | |
| | HCT | | CH3CN | for 1.7 min | | |
| | ultra | | | | | |
| Method D | Waters: | Waters: BEH | A: 95% | 84.2% A for | 0.343 | 6.2 |
| | Acquity | C18 (1.7 μm, | CH₃COONH₄ | 0.49 min, to | 40 | |
| | UPLC ®- | 2.1 × 100 mm) | 7 mM/5% | 10.5% A in | | |
| | DAD and | | CH₃CN, B: | 2.18 min, held for | | |
| | Quattro | | CH₃CN | 1.94 min, back to | | |
| | Micro ™ | | | 84.2% A in | | |
| | | | | 0.73 min, held for | | |
| | | | | 0.73 min. | | |
| Method | Waters: | Waters: BEH | A: 95% | 84.2% A to | 0.343 | 6.1 |
| E | Acquity ® | C18 (1.7 μm | CH₃COON | 10.5% A in | 40 | |
| | H-Class- | 2.1 × 100 mm) | H₄ 7 mM/ | 2.18 min, held | | |
| | DAD and | | 5% CH₃CN, | for 1.96 min, | | |
| | SQD2 ™ | | B: CH₃CN | back to 84.2% | | |
| | | | | A in 0.73 min, | | |
| | | | | held for 0.73 | | |
| | | | | min. | | |

If a compound is a mixture of isomers which give different peaks in the LCMS method, only the retention time of the main component would be given in the LCMS table.

Characterising Data Table

| Com- | | LCMS | | | | |
|---|---|---|---|---|---|---|
| pound | Melting Point | Rt | UV | | BPM1/ | LCMS |
| No | (Kofler or DSC) | min | Area % | MW | BPM2 | Method |
| 1 | 243.7° C. | 10.7 | 98.82 | 435.40 | 436 | Method B |
| 2 | 244.8° C. (DSC 1 | 10 | 99.7 | 449.1 | 450 | Method A |
| | Mettler Toledo | | | | | |
| | 5° C./min | | | | | |
| 3 | 257.8° C. (DSC 1 | 11.2 | 99.3 | 434.1 | 435 | Method B |
| | Mettler Toledo | | | | | |
| | 5° C./min | | | | | |
| 4 | 337.2° C. (DSC | 2.23 | 98.4 | 351.4 | 352.5/ | Method E |
| | Mettler Toledo | | | | 350.5 | |
| | 10° C./min | | | | | |
| 5 | 229.9° C. (DSC | 2.77 | 98.4 | 435.4 | 436.2/ | Method D |
| | Mettler Toledo | | | | 434.2 | |
| | 10° C./min | | | | | |
| 6 | 306.4° C. (DSC | 2.75 | 100 | 435.4 | 436.2/ | Method D |
| | Mettler Toledo | | | | 434.2 | |
| | 10° C./min | | | | | |
| 7 | | 2.69 | 99.8 | 520.2 | 521.3/ | Method D |
| | | | | | 519.3 | |

### Pharmacological Examples

In the tests described below, individual compounds of the invention/examples (or combinations containing such compounds, for instance cytochrome bd inhibitors of the invention/examples in combination with one or more other inhibitor(s) of a (different) target of the electron transport chain of mycobacteria, as described herein) may be tested. For instance, in Tests 1 to 4, combinations may be tested (e.g. combinations of test cytochrome bd compounds with known cytochrome be inhibitors, such as Q203 and Compound X). Where a control cytochrome bd compound is employed, then CK-2-63 is employed.

The compound Q203 (cytochrome bc1 inhibitor) may be prepared in accordance with the procedures in J. Medicinal Chemistry, 2014, 57 (12), pp 5293-5305, as well as, in WO 2011/113606 (see Compound 289 “6-chloro-2-ethyl-N-(4-(4-(4-(trifluoromethoxy)phenyl)piperidin-1-yl)benzyl)imidazo[1,2-a]pyridine-3-carboxamide”).

Compound X is 6-chloro-2-ethyl-N-({4-[2-(trifluoromethanesulfonyl)-2-azaspiro[3.3]heptan-6-yl]phenyl}methyl)imidazo[1,2-a]pyridine-3-carboxamide, which is described as Compound 154 of WO 2017/001660 and may be prepared according to the procedures described therein.

CK-2-63 may be prepared in accordance with the procedures disclosed in WO 2017/103615 (see experimental and the disclosures therein, referring to WO 2012/2069856, where an experimental procedure is provided for “3-methyl-2-(4-(4-(trifluoromethoxy)phenoxy)phenyl)quinolin-4(1H)-one”).

MIC Determination Against M. tuberculosis: Test 1

Test compounds and reference compounds were dissolved in DMSO and 1 μl of solution was spotted per well in 96 well plates at 200× the final concentration. Column 1 and column 12 were left compound-free, and from column 2 to 11 compound concentration was diluted 3-fold. Frozen stocks of Mycobacterium tuberculosis strain EH4.0 expressing green-fluorescent protein (GFP) were previously prepared and titrated. To prepare the inoculum, 1 vial of frozen bacterial stock was thawed to room temperature and diluted to 5×10 exp5 colony forming units per ml in 7H9 broth. 200 μl of inoculum, which corresponds to 1×10 exp5 colony forming units, were transferred per well to the whole plate, except column 12. 200 μl 7H9 broth were transferred to wells of column 12. Plates were incubated at 37° C. in plastic bags to prevent evaporation. After 7 days, fluorescence was measured on a Gemini EM Microplate Reader with 485 excitation and 538 nm emission wavelengths and IC₅₀ and/or pIC₅₀ values (or the like, e.g. IC₅₀, IC₉₀, pIC₉₀, etc) were (or may be) calculated.

MIC Determination Against M. tuberculosis: Test 2

Appropriate solutions of experimental and reference compounds were made in 96 well plates with 7H9 medium. Samples of Mycobacterium tuberculosis strain H37Rv were taken from cultures in logarithmic growth phase. These were first diluted to obtain an optical density of 0.3 at 600 nm wavelength and then diluted 1/100, resulting in an inoculum of approximately 5×10 exp5 colony forming units per ml. 100p of inoculum, which corresponds to 5×10 exp4 colony forming units, were transferred per well to the whole plate, except column 12. Plates were incubated at 37° C. in plastic bags to prevent evaporation. After 7 days, resazurin was added to all wells. Two days later, fluorescence was measured on a Gemini EM Microplate Reader with 543 excitation and 590 nm emission wavelengths and MIC₅₀ and/or pIC₅₀ values (or the like, e.g. IC₅₀, IC₉₀, pIC₉₀, etc) were (or may be) calculated.

Time Kill Kinetics Assays: Test 3

Bactericidal or bacteriostatic activity of the compounds can be determined in a time kill kinetic assay using the broth dilution method. In this assay, the starting inoculum of M. tuberculosis (strain H37Rv and H37Ra) is 10⁶ CFU/ml in Middlebrook (1×) 7H9 broth. The test compounds (cyt bd inhibitors) are tested in combination with a cyt be inhibitor (for example Q203 or Compound X) at the concentration ranging from 10-30 μM to 0.9-0.3 μM respectively. Tubes receiving no antibacterial agent constitute the culture growth control. The tubes containing the microorganism and the test compounds are incubated at 37° C. After 0, 1, 4, 7, 14 and 21 days of incubation samples are removed for determination of viable counts by serial dilution (10⁰ to 10⁻⁶) in Middlebrook 7H9 medium and plating (100 μl) on Middlebrook 7H111 agar. The plates are incubated at 37° C. for 21 days and the number of colonies are determined. Killing curves can be constructed by plotting the log₁₀CFU per ml versus time. A bactericidal effect of a cytochrome be and cytochrome bd inhibitor (either alone or in combination) is commonly defined as 2-log₁₀ decrease (decrease in CFU per ml) compared to Day 0. The potential carryover effect of the drugs is limited by using 0.4% charcoal in the agar plates, and by serial dilutions and counting the colonies at highest dilution possible used for plating.

Phenotypic Assay to Determine the O₂ Consumption Rate of Mycobacterium tuberculosis: Test 4

The aim of this assay is to evaluate the O₂ consumption rate of Mycobacterium tuberculosis (Mtb) bacilli after inhibition of cyt bc1 and cyt bd, using extracellular flux technology. Inhibition of cyt bc1 (e.g. using known inhibitors such as Q203 or Compound X) forces the bacillus to use the less energetically efficient terminal oxidase cyt bd. The inhibition of cyt bd will cause a significant decrease O₂ consumption. A sustained decrease of O₂ consumption under membrane potential disrupting conditions, via the addition of the uncoupler CCCP, will show to the efficacy of the cyt bd inhibitor.

The oxygen consumption rate (OCR) of Mtb (stain H37Ra) bacilli adhered to the bottom of a Cell-Tak (BD Biosciences) coated XF cell culture microplate (Agilent), at 5×10⁶ bacilli per well, was measured using the Agilent Seahorse XFe96. Prior to the assay Mtb bacilli are cultured for two days to an OD₆₀₀˜0.7-0.9 in liquid medium, using 7H9 supplemented with 10% and 0.02% Tyloxapol. The assay media used is unbuffered 7H9 only supplemented with 0.2% glucose. For this assay the Compound X (final concentration of 0.9 μM, Compound X), is used to inhibit cyt bc1 and the cyt bd inhibitor, CK-2-63 (final concentration of 10 μM), is used as a positive control. The uncoupler CCCP is used at a final concentration of 1 μM.

In general, four basal OCR measurements are taken before the automatic addition of Compound X, through drug port A of the sensor cartridge, after which seven more OCR measurements are taken to allow enough time for the inhibition of cyt bc1. Next the cyt bd test compounds (final concentration of 10 μM), as well as the positive and negative controls (assay media with a final DMSO concentration of 0.4%), are added (drug port B) followed by seven OCR measurements. Finally, CCCP is added followed by three OCR measurements, this is done twice (drug ports C and D). For the control's measurements are performed in eight replicate wells and for the assay compounds six replicate wells per condition. Compounds are scored for their sustained inhibition of cyt bd in relation to the positive and negative controls.

Further Phenotypic Assay: Using a Cytochrome Bc Knock-Out TB Strain and MIC Determination Against M. tuberculosis: Test 5

Appropriate solutions of experimental and reference compounds were made in 384 well plates with 7H9 medium. Samples of Mycobacterium tuberculosis strain H37Rv ΔctaE-ΔqcrCAB (Nat Commun 10, 4970, 2019, https://doi.org/10.1038/s41467-019-12956-2) were taken from cultures in logarithmic growth phase. These were first diluted to obtain an optical density of 0.4 at 600 nm wavelength and then diluted 1/150, resulting in an inoculum of approximately 5×10 exp5 colony forming units per ml. 30 μl of inoculum, which corresponds to 5×10 exp5 colony forming units, were transferred per well to the whole plate, except columns 23-24. Plates were incubated at 37° C., in an extra humidified incubator, in plastic bags to prevent evaporation. After 10 days, optical density at 620 nm wavelength was measured on an EnVision 2105 Multimode Plate Reader with a Photometric 620/8 excitation filter, and MIC₅₀ and/or pIC₅₀ values (or the like, e.g. IC₅₀, IC₉₀, pIC₉₀, etc) were (or may be) calculated.

### Pharmacological Results

### Biological Data—Example A

Compounds of the invention/examples (or combinations, e.g. compounds of the invention/examples in combination with one or more other inhibitors of a target of the electron transport chain), for example when tested in any of Tests 1 to 3, may display activity.

### Biological Data—Example B

Compounds of the examples were tested in Test 4 described above (in section “Pharmacological Examples”; O₂ consumption rate testing), together with Compound X—a known cytochrome be inhibitor—as described above, and the following results were obtained:

| | (i) % | |
|---|---|---|
| | OCR after | (ii) |
| | cyt bd | % OCR |
| Example | inhibitor | after cccp |
| 1 | 62.4 | 93.2 |
| 2 | 111.8 | 112.0 |
| 3 | 98.5; 98.0 | 196.1; 131.0 |
| 4 | 83.4 | 144.1 |
| 5 | 80.8 | 159.3 |
| 6 | 104.1 | 177.3 |
| 7 | 90.7 | 110.6 |
| 8 | 62.42 | 93.125 |

### Biological Data—Example C

Compounds of the examples are tested in Test 3 (the kill kinetics) described above, obtaining results expressed as a log reduction in CFUs per ml as compared to Day 0

### Biological Data—Example D

Compounds of the examples were re-tested in Test 5 described above, and the following results were obtained:

| Compound | |
|---|---|
| number | pIC₅₀ |
| 4 | <4.000 |
| 5 | <4.000 |
| 2 | 4.037 |
| 3 | <4.000 |
| 1 | 4.556 |

Further Data

The compounds of the invention/examples may have advantages associated with in vitro potency, kill kinetics (i.e. bactericidal effect) in vitro, PK properties, food effect, safety/toxicity (including liver toxicity, coagulation, 5-LO oxygenase), metabolic stability, Ames II negativity, MNT negativity, aqueous based solubility (and ability to formulate) and/or cardiovascular effect e.g. on animals (e.g. anesthetized guinea pig). The data below that was generated/calculated may be obtained using standard methods/assays, for instance that are available in the literature or which may be performed by a supplier (e.g. Microsomal Stability Assay—Cyprotex, Mitochondrial toxicity (Glu/Gal) assay—Cyprotex, as well as literature CYP cocktail inhibition assays).

Mitotoxicity Data:

| | | | Δ | |
|---|---|---|---|---|
| cpd | IC50, | IC50, | IC50, glu/ | |
| number | glu | gal | IC50, gal | Score |
| 8 | [x]200 | [x]10.82 | [x]18.49 | positive |

The score for other compounds was as follows:

Compound 1: positive

Compound 2: positive

Compound 3: negative

Compound 7: negative

Certain compounds of the invention/examples may be found to be advantageous as no mitotoxicity alerts were observed (e.g. in the Glu/Gal assay).

| | Cardio tox |
|---|---|
| Compound | rCaCH (pIC50), rNaCH (IC50), hERG(DOF) (IC50) |
| 5 | >10, 3.8, >10 |
| 2 | >10, 2.6, 1.8 |
| 3 | >10, 1.9, >10 |
| 1 | 3.5, 3.02, 3.98 |

Compounds of the invention/examples, may therefore have the advantage that:
No in vitro cardiotoxicity is observed (for example either due to the CVS results or due to the Glu/Gal assay results, for instance low mitotoxicity (<3 in the Glu/Gal assay indicates no mitotoxicity alerts); and/or
No reactive metabolite formation is observed (e.g. GSH);

for instance as compared to other compounds, for instance prior art compounds.

1. A compound of formula (I): 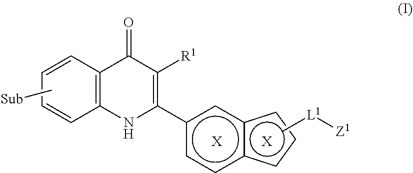
wherein:
R¹ is C₁₋₆ alkyl, —Br, hydrogen or —C(O)N(R^{q1})R^{q2};
R^{q1} and R^{q2} independently are hydrogen or C₁₋₆ alkyl, or are linked together to form a 3-6 membered carbocyclic ring optionally substituted by one or more C₁₋₃ alkyl substituents;
Sub is one or more optional substituents that are halo, —CN, C₁₋₆ alkyl or —O—C₁₋₆ alkyl (wherein the latter two alkyl moieties are optionally substituted by one or more fluoro atoms);
the two “X” rings together are a 9-membered bicyclic heteroaryl ring containing between one and four heteroatoms, and is optionally substituted by one or more substituents that are halo or C₁₋₆ alkyl (itself optionally substituted by one or more fluoro atoms);
L¹ is an optional linker group;
R^{x1} and R^{x2} independently represent are hydrogen or C₁₋₃ alkyl;
Z¹ is one of the following moieties: 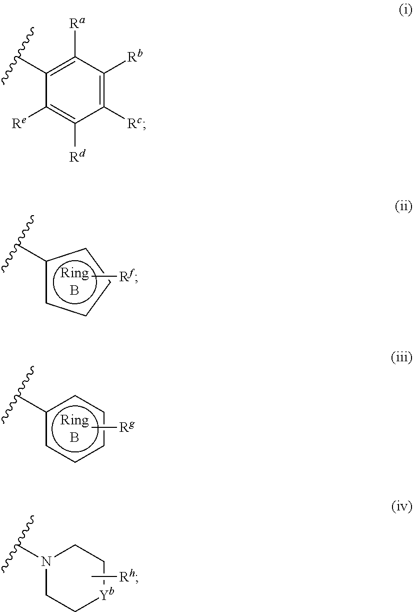
(v) perfluoro C₁₋₃ alkyl;
(vi) —F, —Br, —Cl or —CN;
ring A is a 5-membered aromatic ring containing at least one heteroatom and is optionally substituted by one or more substituents independently that are R^{f};
ring B is a 6-membered aromatic ring containing at least one heteroatom and is optionally substituted by one or more substituents independently that are R^{g};
Y^{b} is —CH₂ or NH, and R^{h} are one or more substituents on the 6-membered N and Y^{b}-containing ring (which R^{h} substituents are optionally present on Y^{b});
R^{a}, R^{b}, R^{c}, R^{d} and R^{e} independently are hydrogen or a substituent that is B¹;
each R^{f}, each R^{g} and each R^{h} (which are optional substituents), when present, independently are a substituent that is B¹;
each B¹ independently is:
(i) halo;
(ii) —R^{d1};
(iii) —OR^{e1};
(iv) —C(O)N(R^{e2})R^{e3}
(v) —SF₅; or
(vi) —N(R^{e4})S(O)₂R^{e5};
R^{d1} is C₁₋₆ alkyl optionally substituted by one or more halo atoms;
R^{e1}, R^{e2}, R^{e3}, R^{e4} and R^{e5} each independently are hydrogen or C₁₋₆ alkyl optionally substituted by one or more fluoro atoms,
or a pharmaceutically-acceptable salt thereof.

2. The compound of claim 1, wherein R¹ is C₁₋₃ alkyl.

3. The compound of claim 1, wherein the “X” rings:
contains at least one nitrogen atom; and/or
contains one, two, three or four heteroatoms in total.

4. The compound of claim 1, wherein the “X” rings is of formula (IA): 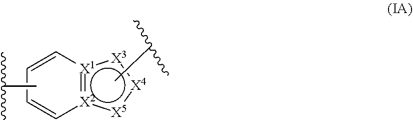
wherein:
one of X¹ and X² is N and the other is C;
the other integers X³, X⁴ and X⁵ are C, CH, or a heteroatom; or
none, any one or two of X³, X⁴ and X⁵ is a heteroatom and the other is C (or CH).

5. The compound of claim 1, wherein:
L¹ is a direct bond, —O—, —OCH₂—, —C(R^{x1})(R^{x2})— or —C(O)—N(H)—CH₂—;
R^{x1} and R^{x2} independently are hydrogen.

6. The compound of claim 1, wherein:
none, one or two of R^{a}, R^{b}, R^{c}, R^{d} and R^{e} is B¹ and the others are hydrogen; or
one of R^{b}, R^{c} and R^{d} is B¹ and the others are hydrogen.

7. The compound of claim 1, wherein B¹ is:
(i) fluoro;
(ii) —OR^{e1};
(iii) C₁₋₃ alkyl substituted by one or more fluoro atom;
(iv) —C(O)N(R^{e2})R^{e3};
(v) —N(R^{e4})S(O)₂R^{e5}; or
(vi) —SF₅.

8. The compound of claim 1, wherein:
R^{e2} and R^{e4} independently are hydrogen;
R^{e1}, R^{e3} and R^{e5} each independently are C₁₋₃ alkyl optionally substituted by one or more fluoro atoms.

9. (canceled)

10. A pharmaceutical composition comprising a pharmaceutically acceptable carrier and, as active ingredient, a therapeutically effective amount of the compound of claim 1.

11. (canceled)

12. (canceled)

13. A method of treatment of tuberculosis, comprising administration of a therapeutically effective amount of the compound of claim 1.

14. A combination of (a) a compound of claim 1, and (b) one or more other anti-tuberculosis agent.

15. A product containing (a) a compound of claim 1, and (b) one or more other anti-tuberculosis agent, as a combined preparation for simultaneous, separate or sequential use in the treatment of a bacterial infection.

16. (canceled)

17. (canceled)

18. A method of treatment of tuberculosis, comprising administration of a therapeutically effective amount of a combination of claim 14.

19. A method of enhancement of activity of another anti-tuberculosis agent, comprising administering the compound of claim 1 in combination with the another anti-tuberculosis agent.

20. A process for the preparation of a compound of formula (I) of claim 1, comprising:
(i) conversion of a compound of formula (II), 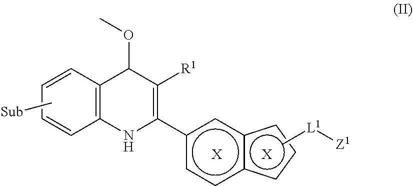
by reaction with an appropriate reagent; or
(ii) reaction of a compound of formula (III), 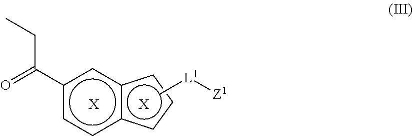
with a compound of formula (IV),

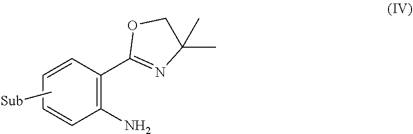

21. The compound of claim 1, wherein:
the two “X” rings together form a 6-membered aromatic ring fused to another 5-membered aromatic ring; and/or
the 9-membered bicyclic heteroaryl ring contains one to four heteroatoms that are nitrogen oxygen or sulfur; and/or
L¹ is a direct bond, —O—, —OCH₂—, —C(R^{x1})(R^{x2})— or —C(O)—N(H)—CH₂—; and/or
Z1 is CF₃; and/or
Ring A contains at last one nitrogen atom; and/or
Ring B contains at last one nitrogen atom; and/or
R^{d1} is optionally substituted by one or more fluoro atoms.

22. The compound of claim 2, wherein R¹ is methyl.

23. The compound of claim 3, wherein the “X” rings contain at least one nitrogen at the ring junction.

24. The compound of claim 4 wherein the “X” rings are

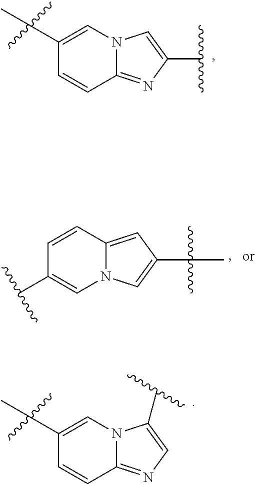 [SMILES: *c1ccc2nc(*)cn2c1 ; *c1ccc2cc(*)cn2c1 ; *c1ccc2ncc(*)n2c1]

25. The compound of claim 4, wherein:
X³, X⁴ and X⁵ are C, CH, N, O or S; or
one or two of X³, X⁴ and X⁵ is a heteroatom; or
one or two of X³, X⁴ and X⁵ is N, O, or S.

26. The compound of claim 6, wherein
one or two of R^{a}, R^{b}, R^{c}, R^{d} and R^{e} is B¹; or
one of R^{a}, R^{b}, R^{c}, R^{d} and R^{e} is B¹; or
one of R^{b}, R^{c} and R^{d} is B¹; or
R^{c} is B¹.

27. The compound of claim 8, wherein R^{e1}, R^{e3} and R^{e5} are methyl.

28. The combination of claim 14, wherein the other anti-tuberculosis agent is an inhibitor of the electron transport chain of mycobacteria.

29. The combination of claim 28, wherein the other anti-tuberculosis agent is a cytochrome be inhibitor, an ATP synthase inhibitor, a NDH2 inhibitor, or an inhibitor of the menaquinone synthesis pathway.

30. The combination of claim 29, wherein the inhibitor of the menaquinone synthesis pathway is a MenG inhibitor.

31. The product of claim 15, wherein the other anti-tuberculosis agent is an inhibitor of the electron transport chain of mycobacteria.

32. The combination of claim 31, wherein the other anti-tuberculosis agent is a cytochrome be inhibitor, an ATP synthase inhibitor, a NDH2 inhibitor, or an inhibitor of the menaquinone synthesis pathway.

33. The combination of claim 32, wherein the inhibitor of the menaquinone synthesis pathway is a MenG inhibitor.

34. The process of claim 20, wherein the appropriate reagent is BBr₃ or NaSCH₃.
